(19) United States
(12) Patent Application Publication
(10) Pub. No.: US 2017/0027985 A1
(43) Pub. Date: Feb. 2, 2017

(54) NUTRITIONAL COMPOSITIONS

(71) Applicant: Cambrooke Therapeutics, Inc., Ayer, MA (US)

(72) Inventors: Susan Gingrich, Hamilton, MA (US); Kurt Olson, Chicago, IL (US)

(73) Assignee: Cambrooke Therapeutics, Inc., Ayer, MA (US)

(21) Appl. No.: 15/200,661

(22) Filed: Jul. 1, 2016

Related U.S. Application Data
(60) Provisional application No. 62/189,145, filed on Jul. 6, 2015.
(60) Provisional application No. 62/188,208, filed on Jul. 2, 2015.

(51) Int. Cl. A61K 33/42; A61K 33/30; A61K 33/06; A61K 31/122; A61K 31/733; A61K 31/4172; A61K 31/198; A61K 31/405; A61K 38/16; A61K 31/70; A61K 31/205; A61K 31/145; A61K 31/07; A61K 31/355; A61K 31/4188; A61K 31/519; A61K 31/455; A61K 31/197; A61K 31/51; A61K 31/714; A61K 31/525; A61K 31/4415; A61K 31/375; A61K 33/24; A61K 33/34; A61K 33/18; A61K 33/26; A61K 33/32; A61K 33/00; A61K 33/04; A23L 33/155; A23L 33/16; A23L 33/175; A61K 31/593

(52) U.S. Cl. CPC A61K 33/42; A61K 31/593; A61K 33/30; A61K 33/06; A61K 31/122; A61K 31/733; A61K 31/4172; A61K 31/198; A61K 31/405; A61K 38/16; A61K 31/70; A61K 31/205; A61K 31/145; A61K 31/07; A61K 31/355; A61K 31/4188; A61K 31/519; A61K 31/455; A61K 31/197; A61K 31/51; A61K 31/714; A61K 31/525; A61K 31/4415; A61K 31/375; A61K 33/24; A61K 33/34; A61K 33/18; A61K 33/26; A61K 33/32; A61K 33/00; A61K 33/04; A23L 33/155; A23L 33/16; A23L 33/175; A23V 2002/00

(57) ABSTRACT

In some aspects, the disclosure relates to compositions useful for the dietary management of inborn errors of metabolism (e.g., PKU, MSUD, HCU, IVA, and MMA/PA). In some embodiments, the disclosure provides a composition comprising a protein source, a fat source, a carbohydrate source, at least one vitamin, and at least one mineral, wherein the protein source comprises at least six free amino acids. In some aspects the disclosure relates to compositions that support bone health.

### RELATED APPLICATIONS

This application claims the benefit under 35 U.S.C. 119(e) of U.S. provisional patent application U.S. Ser. No. 62/188,208, filed on Jul. 2, 2015, entitled “Nutritional Compositions”, and U.S. provisional patent application U.S. Ser. No. 62/189,145, filed on Jul. 6, 2016, entitled “Nutritional Compositions.” The teachings of both applications are incorporated by reference herein.

### BACKGROUND OF INVENTION

Inborn errors of metabolism are a group of inherited disorders characterized by defective function of one or more metabolic enzymes. Several inborn errors of metabolism are related to enzymes that metabolize amino acids. In these disorders, a lack of enzymatic activity causes an inability to fully metabolize proteins or peptides, leading to an accumulation of toxic byproducts and reduced ability to synthesize essential compounds. For example, Phenylketonuria (PKU) is caused by a mutation that reduces or abrogates activity of the phenylalanine hydroxylase (PAH) gene. Impaired metabolism leads to a toxic buildup of phenylalanine (Phe) in the brain, resulting in intellectual disability and seizures. Inborn errors of metabolism are also often related to secondary medical issues, such as low bone mineral density.

Inborn errors of metabolism related to amino acid metabolism can be managed by a controlled diet that reduces or excludes the amino acid that the patient is unable to break down (e.g., a diet lacking or low in Phe for a PKU patient). Generally, the protein source provided by these diets is a mixture of elemental amino acids. Controlled diets can be administered in the form of nutritional formulas, which can be reconstituted from powdered mixes or provided in a ready to drink (RTD) liquid format. However, patient compliance with controlled diets based on nutritional formulas is an issue, with some patients citing unacceptable taste of nutritional formulas as a reason for non-compliance.

Accordingly, there is a need to develop novel nutritional formulas having acceptable palatability and improved nutrient profiles for the dietary management of inborn errors of metabolism.

### SUMMARY OF INVENTION

Described herein are compositions (e.g., amino acid-based formulas) that are useful for the dietary management of inborn errors of metabolism. In some embodiments, the compositions are nutritionally complete, support patient bone health and have an acceptable taste.

In some aspects, the disclosure relates to compositions for the support of bone health (bone health composition) in patients with inborn errors of metabolism. Bone health compositions can be solid (e.g., a powder) or liquid. In some embodiments, bone health compositions comprise Vitamin D, calcium, phosphorus, zinc, magnesium, and Vitamin K. In some embodiments, Vitamin K is a combination of Vitamin K1 and Vitamin K2. In some embodiments, equal amounts (e.g., by gram weight) of Vitamin K1 and Vitamin K2 are present in the composition. In some embodiments, bone health compositions further comprise a dietary fiber. In some embodiments, the dietary fiber is inulin.

The amount of each component in a bone health composition can be expressed as a percentage of the total weight of the bone health composition or as the weight of each component in the amount of bone health composition that is included per serving of nutritional composition. Typically the amount of bone health composition is from about 1.5 g to about 4.0 g per serving. The percentage of bone health blend in a total serving ranges from about 0.43 to about 0.68. In some embodiments, bone health compositions further comprise a protein source and the amount of each component in the composition is expressed as weight per g protein equivalent (PE) (e.g., per 20 g PE). In some embodiments, a protein source comprises one or more amino acids selected from the group consisting of: histidine, isoleucine, leucine, lysine, methionine, phenylalanine, threonine, tryptophan, and valine. Examples of ranges for each component of a bone health composition are described in Table 1. In some embodiments, a bone health composition is a component in a vitamin and mineral mixture, for example as shown in Table 2 or Table 7.

In some aspects, the disclosure provides nutritional compositions for dietary management of inborn errors of metabolism comprising components described herein. The inborn error of metabolism can be Phenylketonuria (PKU), Maple Syrup Urine Disease (MSUD), Homocystinuria (HCU), Isovaleric acidaemia (IVA), and Methylmalonic acidaemia/Propionic acidaemia (MMA/PA). In some embodiments, nutritional compositions comprise a protein source, a fat source, a carbohydrate source, the bone health composition, wherein the protein source comprises at least six free amino acids. The nutritional compositions are liquids and in some embodiments, the pH of a nutritional composition is between about pH 5.0 and about pH 6.0.

In some embodiments, the at least six free amino acids are selected from the group consisting of: histidine, isoleucine, leucine, methionine, phenylalanine, threonine, tryptophan, and valine. In some embodiments, nutritional compositions further comprise at least one amino acid selected from the group consisting of glycine, alanine, arginine, asparagine, aspartic acid, cystine, glutamic acid, glutamine, proline, serine, and tyrosine. In some embodiments, the protein source is a mixture of amino acids (e.g., a blend of amino acids), for example as shown in Table 4 or Table 10.

The amount of each amino acid in a nutritional composition can be expressed as weight per g protein equivalent (PE) (e.g., per 20 g PE), or as weight of each component per serving. In some embodiments, a serving of liquid nutritional composition is about 8.5 fluid oz (i.e., about 250 mL).

The liquid nutritional formula may contain additional components, for example carnitine, taurine, Docosahexaenoic acid (DHA), alone or in combination (e.g., carnitine and taurine; carnitine, taurine, and DHA; carnitine and DHA; or taurine and DHA).

In some embodiments, the nutritional composition comprises: Vitamin A, Vitamin D, Vitamin E, biotin, folic acid, niacin, pantothenic acid, Vitamin B1, Vitamin B12, Vitamin B2, Vitamin B6, Vitamin C, and Vitamin K and calcium, chromium, copper, iodine, iron, magnesium, manganese, molybdenum, phosphorus, potassium, selenium, sodium, and zinc. In some embodiments, the nutritional composition includes a vitamin and mineral blend, for example as represented in Table 2 or Table 7. The amount of each vitamin or mineral in a nutritional composition for dietary management of inborn errors of metabolism can be expressed as a percentage of the total weight of the composition, the weight of each vitamin or mineral per serving, or the weight of each vitamin or mineral per g PE (e.g., per 20 g PE).

In some aspects, the disclosure relates to a nutritional composition for dietary management of an inborn error of metabolism, comprising the components set forth in Table 5. In some embodiments, the amino acid blend is selected from the blends in Table 4 or Table 10. In some embodiments, the amino acid blend is selected from the group consisting of Blend 1, Blend 2, Blend 3, Blend 4 or Blend 5 in Table 4, and Blend 1, Blend 2, Blend 3, Blend 4 or Blend 5 in Table 10. In some embodiments, a nutritional composition for dietary management of an inborn error of metabolism provides (per 8.5 fluid oz.) about 15, about 16 g PE, about 17 g PE, about 18 g PE, about 19 g PE, about 20 g PE, or about 21 g PE.

### BRIEF DESCRIPTION OF THE DRAWINGS

FIG. 1 shows non-limiting embodiments of nutritional compositions for dietary management of inborn errors of metabolism comprising components described by the disclosure.

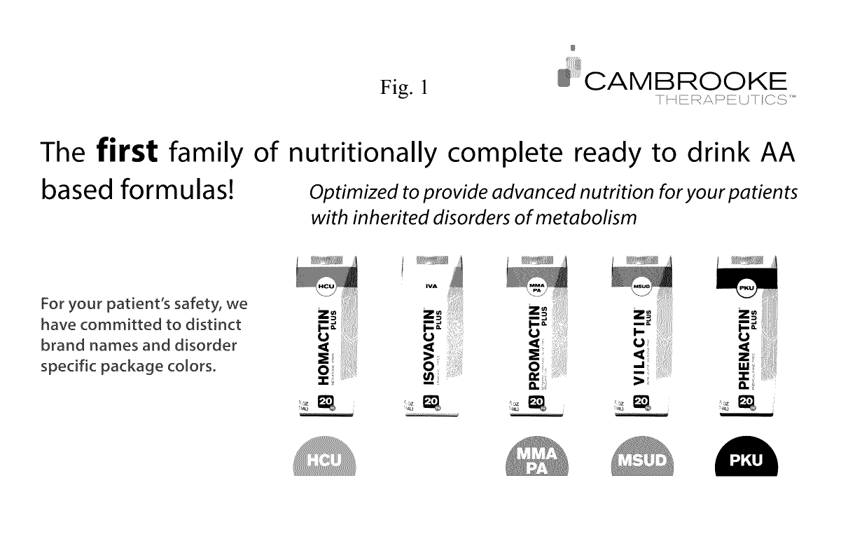

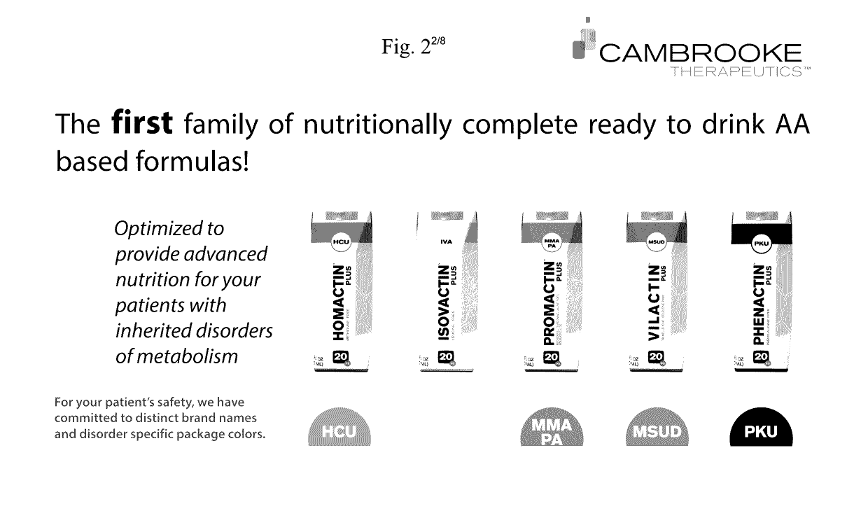

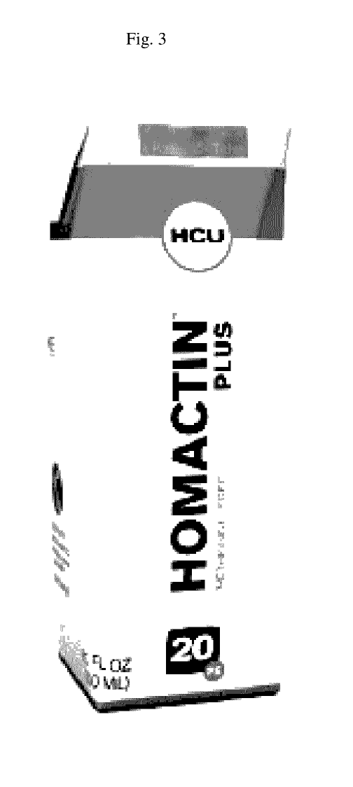

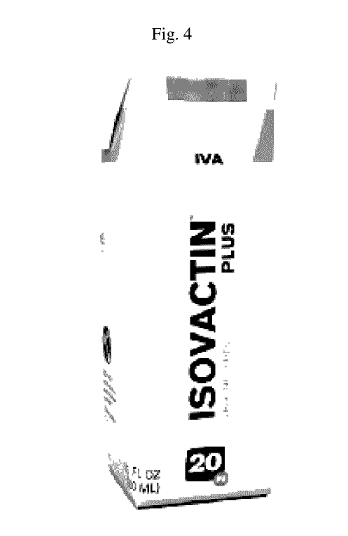

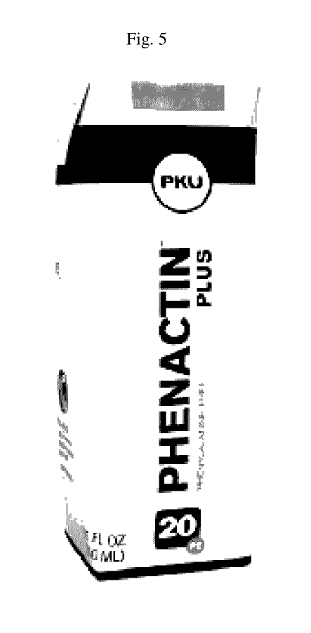

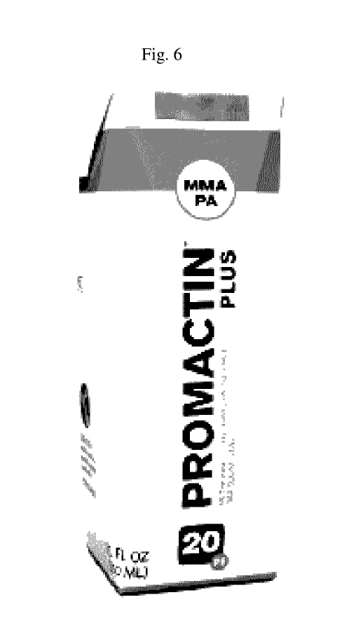

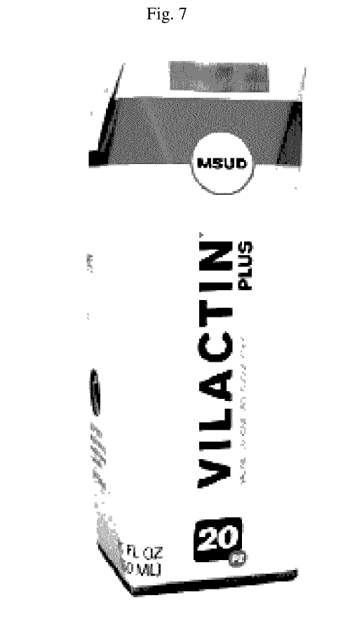

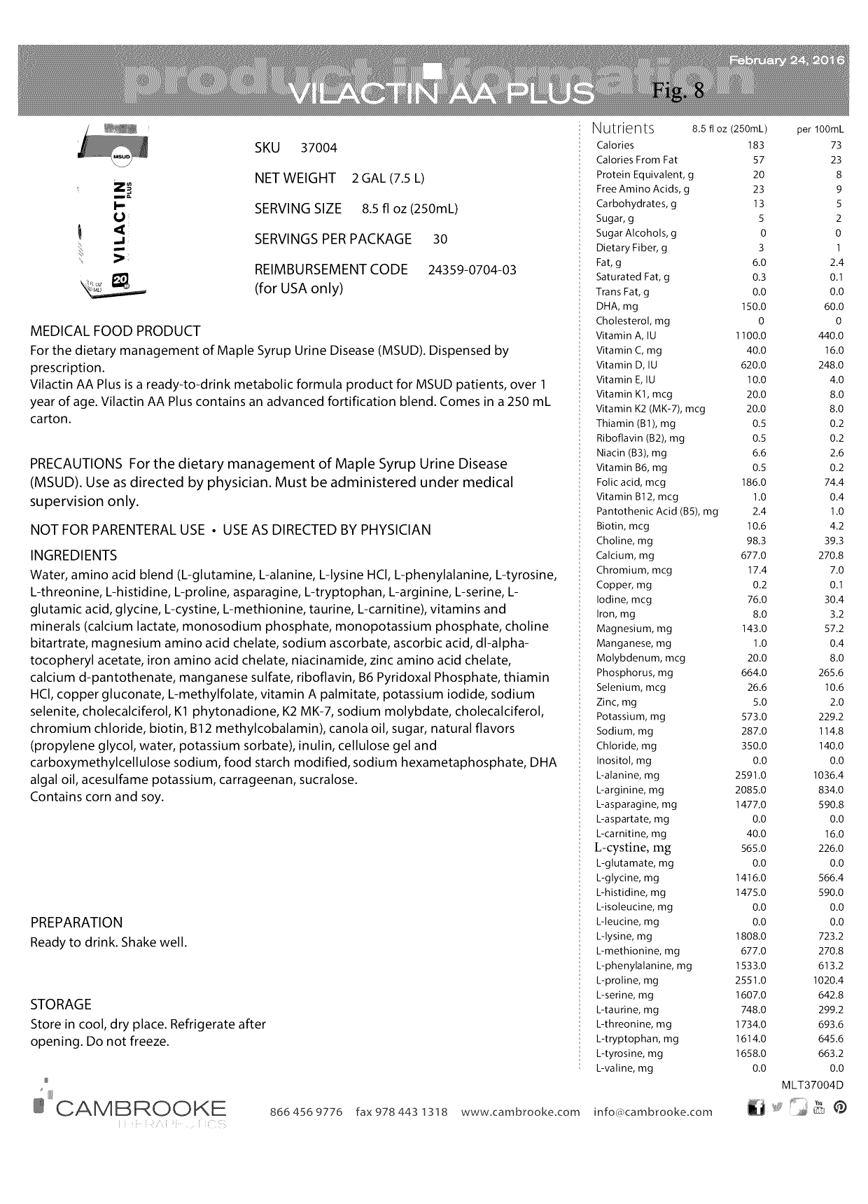

OPTIMIZED PROFILE FEATURES AND BENEFITS
Nutritionally complete plus EFAs—so you are meeting not just their protein needs
20 g PE/serving—a concentrated source of protein to help with adherence
Bone Health Blend optimized to promote bone health:
Per serving:
   Vitamin D 620 IU—highest level compared to other AA formulas
   Calcium 700 mg
   Phosphorus 695 mg
   Zn 5 mg
   Mg 160 mg
   Vitamin K (K1, K2) 40 mcg, levels optimized for bone health¹
   Inulin 2 g, to aid in calcium absorption² (excludes Promactin)
   DHA 150 mg/serving—to support brain and eye development
   3 g Pre-biotic fiber/serving to support digestive health (excludes Promactin)
   pH 5.8, Lower acidity than other AA formulas—gentler on digestion and overall milder taste
   Great tasting Berry flavor—smooth taste unlike other AA RTD products
   No artificial flavoring or coloring—requested by you and your patients
   Convenient ready to drink—making it easy to fit into your busy life FOR A SAMPLE call 866 456 9776, opt 2 or complete the online form: www.samples.cambrooke.com
CAMBROOKEcare™
CAMBROOKEcare is available to anyone needing assistance with:
low protein food coverage support
metabolic formula coverage support
coverage assistance support services
co-pay assistance
coding and billing information for patients and healthcare providers
insurance direct billing services
supplier sourcing (pharmacy, durable medical equipment [DME], etc. . . . )
CONTACT OUR COVERAGE SPECIALISTS:
866 383 9455
reimbursement@cambrooke.com
CAMBROOKEcare™ is a full service coverage and reimbursement assistance program offered through Cambrooke Therapeutics™

### REFERENCES

1. Marieke J, et al. (2008). Vitamin K status is associated with childhood bone mineral content. British Journal of Nutrition, 100, 852-858.
2. Scholz-Ahrens, K, Schrezenmeir, J. Inulin, oligofructose and mineral metabolism—experimental data and mechanism.
CAMBROOKEcare™ 866 383 9455 reimbursement@cambrooke.com cambrookecare.com, © Cambrooke Therapeutics, Inc. All Rights Reserved R062515

FIG. 2 shows non-limiting embodiments of nutritional compositions for dietary management of inborn errors of metabolism comprising components described by the disclosure.

OPTIMIZED PROFILE FEATURES AND BENEFITS
Nutritionally complete plus EFAs—so you are meeting not just their protein needs
20 g PE/serving—a concentrated source of protein to help with adherence
Bone Health Blend optimized to promote bone health:
Per serving:
   Vitamin D 620 IU—highest level compared to other AA formulas
   Calcium 700 mg
   Phosphorus 695 mg
   Zn 5 mg
   Mg 160 mg
   Vitamin K (K1, K2) 40 mcg, levels optimized for bone health′
   Inulin 2 g, to aid in calcium absorption² (excludes Promactin)
DHA 150 mg/serving—to support brain and eye development
3 g Pre-biotic fiber/serving to support digestive health (excludes Promactin)
pH 5.8, Lower acidity than other AA formulas—gentler on digestion and overall milder taste
Great tasting Berry flavor—smooth taste unlike other AA RTD products
No artificial flavoring or coloring—requested by you and your patients
Convenient ready to drink—making it easy to fit into your busy life
FOR A SAMPLE
call 866 456 9776, opt 2 or complete the online form:
www.samples.cambrooke.com
CAMBROOKEcare™
CAMBROOKEcare is available to anyone needing assistance with:
   low protein food coverage support
   metabolic formula coverage support
   coverage assistance support services
   co-pay assistance
   coding and billing information for patients and healthcare providers
   insurance direct billing services
   supplier sourcing (pharmacy, durable medical equipment [DME], etc. . . . )
CONTACT OUR COVERAGE SPECIALISTS:
866 383 9455
reimbursement@cambrooke.com
CAMBROOKEcare™ is a full service coverage and reimbursement assistance program offered through Cambrooke Therapeutics™

### REFERENCES

1. Marieke J, et al. (2008). Vitamin K status is associated with childhood bone mineral content. British Journal of Nutrition, 100, 852-858.
2. Scholz-Ahrens, K, Schrezenmeir, J. Inulin, oligofructose and mineral metabolism—experimental data and mechanism.
CAMBROOKEcare™ 866 383 9455 reimbursement@cambrooke.com cambrookecare.com
© Cambrooke Therapeutics, Inc. All Rights Reserved R062515

FIG. 3 shows a non-limiting embodiment of a product label for a nutritional composition for dietary management of Homocystinurea (HCU).

SKU 37001

NET WEIGHT 2 GAL (7.5 L)

SERVING SIZE 8.5 fl oz (250 mL)

SERVINGS PER PACKAGE 30

REIMBURSEMENT CODE 24359-0701-03 (for USA only)

### MEDICAL FOOD PRODUCT

For the dietary management of Homocystinurea (HCU). Dispensed by prescription. Homactin AA Plus is a ready-to-drink metabolic formula product for Homocystinurea patients, over 1 year of age. Homactin contains an advanced fortification blend. Product comes in a 250 mL carton.
PRECAUTIONS For the dietary management of Homocystinuria (HCU). Use as directed by physician. Must be administered under medical supervision only.

### NOT FOR PARENTERAL USE•USE AS DIRECTED BY PHYSICIAN

### INGREDIENTS

Water, amino acid blend (L-leucine, L-aspartic, L-proline, L-lysine HCl, L-valine, L-arginine, glycine, L-isoleucine, L-tyrosine, L-serine, L-threonine, L-phenylalanine, L-alanine, L-histidine, L-cystine, L-tryptophan, taurine, L-carnitine, maltodextrin), vitamins and minerals (calcium lactate, monosodium phosphate, monopotassium phosphate, choline bitartrate, magnesium amino acid chelate, sodium ascorbate, ascorbic acid, dl-alpha-tocopheryl acetate, Fe amino acid chelate, niacinamide, zinc amino acid chelate, calcium d-pantothenate, manganese sulfate, riboflavin, B6 Pyrodixine HCl, thiamin HCl, copper gluconate, folic acid, vitamin A palmitate, potassium iodide, sodium selenite, cholecalciferol, K1 phytonadione, K2 MK-7, sodium molybdate, cholecalciferol, chromium chloride, biotin, B12 cyanocobalamin), canola oil, sugar, natural flavors (propylene glycol, water, potassium sorbate), inulin, cellulose gel and carboxymethylcellulose sodium, food starch modified, sodium hexametaphosphate, DHA algal oil, acesulfame potassium, carrageenan, sucralose. Contains corn and soy.

### PREPARATION

Ready to drink. Shake well.

### STORAGE

Store in cool, dry place. Refrigerate after opening. Do not freeze.

### TO REQUEST A SAMPLE

Call 866 456 9776, opt 2 or complete form: www.samples.cambrooke.com

### HOMACTIN AA PLUS

| Nutrients | 8.5 fl oz (250 mL) | Per 100 mL |
|---|---|---|
| Calories | 186 | 74 |
| Calories From Fat | 57 | 23 |
| Protein Equivalent, g | 20 | 8 |
| Free Amino Acids, g | 23 | 9 |
| Carbohydrates, g | 13 | 5 |
| Sugar, g | 5 | 2 |
| Sugar Alcohols, g | 0 | 0 |
| Dietary Fiber, g | 2.8 | 1 |
| Fat, g | 6 | 2.4 |
| Saturated Fat, g | 0.5 | 0.2 |
| Trans Fat, g | 0.0 | 0.0 |
| DHA, mg | 150.0 | 60.0 |
| Cholesterol, mg | 0.4 | 0 |
| Vitamin A, IU | 1100.0 | 440.0 |
| Vitamin C, mg | 40.0 | 16.0 |
| Vitamin D, IU | 620.0 | 248.0 |
| Vitamin E, IU | 10.0 | 4.0 |
| Vitamin K1, mcg | 20.0 | 8.0 |
| Vitamin K2 (MK-7), mcg | 20.0 | 8.0 |
| Thiamin (B1), mg | 0.5 | 0.2 |
| Riboflavin (B2), mg | 0.5 | 0.2 |
| Niacin (B3), mg | 6.6 | 2.6 |
| Vitamin B6, mg | 0.5 | 0.2 |
| Folic Acid, mcg | 186.0 | 74.4 |
| Vitamin B12, mcg | 1.0 | 0.4 |
| Pantothenic Acid (B5), mg | 2.4 | 1.0 |
| Biotin, mcg | 10.6 | 4.2 |
| Choline, mg | 209.0 | 83.6 |
| Calcium, mg | 677.0 | 270.8 |
| Chromium, mcg | 17.3 | 6.9 |
| Copper, mg | 0.2 | 0.1 |
| Iodine, mcg | 76.0 | 30.4 |
| Iron, mg | 8.0 | 3.2 |
| Magnesium, mg | 143.0 | 57.2 |
| Manganese, mg | 1.0 | 0.4 |
| Molybdenum, mcg | 20.0 | 8.0 |
| Phosphorus, mg | 664.0 | 265.6 |
| Selenium, mcg | 26.6 | 10.6 |
| Zinc, mg | 5.0 | 2.0 |
| Potassium, mg | 573.0 | 229.2 |
| Sodium, mg | 287.0 | 114.8 |
| Chloride, mg | 463.0 | 185.2 |
| Inositol, mg | 0.0 | 0.0 |
| L-alanine, mg | 1070.0 | 428.0 |
| L-arginine, mg | 1550.0 | 620.0 |
| L-asparagine, mg | 0.0 | 0.0 |
| L-aspartate, mg | 1930.0 | 772.0 |
| L-carnitine, mg | 21.0 | 8.4 |
| L-cystine, mg | 393.2 | 157.3 |
| L-glutamate, mg | 0.0 | 0.0 |
| L-glycine, mg | 1500.0 | 600.0 |
| L-histidine, mg | 930.0 | 372.0 |
| L-isoleucine, mg | 1430.0 | 572.0 |
| L-leucine, mg | 2450.0 | 980.0 |
| L-lysine, mg | 1620.0 | 648.0 |
| L-methionine, mg | 0.0 | 0.0 |
| L-phenylalanine, mg | 1080.0 | 432.0 |
| L-proline, mg | 1760.0 | 704.0 |
| L-serine, mg | 1250.0 | 500.0 |
| L-taurine, mg | 50.0 | 20.0 |
| L-threonine, mg | 1140.0 | 456.0 |
| L-tryptophan, mg | 480.0 | 192.0 |
| L-tyrosine, mg | 786.4 | 314.6 |
| L-valine, mg | 1560.0 | 624.0 |

FIG. 4 shows a non-limiting embodiment of a product label for a nutritional composition for dietary management of Isovaleric Acidemia (IVA).

SKU 37002

NET WEIGHT 2 GAL (7.5 L)

SERVING SIZE 8.5 fl oz (250 mL)

SERVINGS PER PACKAGE 30

REIMBURSEMENT CODE 24359-0702-03 (for USA only)

### MEDICAL FOOD PRODUCT

For the dietary management of Isovaleric Acidemia. Dispensed by prescription. Isovactin AA Plus is a ready-to-drink metabolic formula product for Isovaleric Acidemia patients, over 1 year of age. Isovactin contains an advanced fortification blend. Product comes in a 250 mL carton.
PRECAUTIONS For the dietary management of Isovaleric Acidemia (IVA) and other disorders of leucine metabolism. Use as directed by physician. Must be administered under medical supervision only.

### NOT FOR PARENTERAL USE•USE AS DIRECTED BY PHYSICIAN

### INGREDIENTS

Water, amino acid blend (L-alanine, glycine, L-proline, L-arginine, L-lysine, L-tyrosine, L-phenylalanine, L-serine, L-carnitine, L-aspartic acid, L-threonine, L-valine, L-isoleucine, L-histidine, L-menthionine, L-tryptophan, L-cystine, taurine), vitamins and minerals (calcium lactate, monosodium phosphate, monopotassium phosphate, choline bitartrate, magnesium amino acid chelate, sodium ascorbate, ascorbic acid, dl-alpha-tocopheryl acetate, Fe amino acid chelate, niacinamide, zinc amino acid chelate, calcium d-pantothenate, manganese sulfate, riboflavin, B6 Pyridoxal 5′ Phosphate, thiamin HCl, copper gluconate, L-methylfolate, vitamin A palmitate, potassium iodide, sodium selenite, cholecalciferol, K1 phytonadione, K2 MK-7, sodium molybdate, cholecalciferol, chromium chloride, biotin, B12 methylcobalamin), canola oil, sugar, natural flavors (propylene glycol, water, potassium sorbate), inulin, cellulose gel and carboxymethylcellulose sodium, food starch modified, sodium hexametaphosphate, DHA algal oil, acesulfame potassium, carrageenan, sucralose. Contains corn and soy.

### PREPARATION

Ready to drink. Shake well.

### STORAGE

Store in cool, dry place. Refrigerate after opening. Do not freeze.

### TO REQUEST A SAMPLE

Call 866 456 9776, opt 2 or complete form: www.samples.cambrooke.com

### ISOVACTIN AA PLUS

| Nutrients | 8.5 fl oz (250 mL) | Per 100 mL |
|---|---|---|
| Calories | 186 | 74 |
| Calories From Fat | 57 | 23 |
| Protein Equivalent, g | 20 | 8 |
| Free Amino Acids, g | 22 | 9 |
| Carbohydrates, g | 13 | 5 |
| Sugar, g | 5 | 2 |
| Sugar Alcohols, g | 0 | 0 |
| Dietary Fiber, g | 2.8 | 1 |
| Fat, g | 6 | 2.4 |
| Saturated Fat, g | 0.5 | 0.2 |
| Trans Fat, g | 0.0 | 0.0 |
| DHA, mg | 150.0 | 60.0 |
| Cholesterol, mg | 0.4 | 0 |
| Vitamin A, IU | 1100.0 | 440.0 |
| Vitamin C, mg | 40.0 | 16.0 |
| Vitamin D, IU | 620.0 | 248.0 |
| Vitamin E, IU | 10.0 | 4.0 |
| Vitamin K1, mcg | 20.0 | 8.0 |
| Vitamin K2 (MK-7), mcg | 20.0 | 8.0 |
| Thiamin (B1), mg | 0.5 | 0.2 |
| Riboflavin (B2), mg | 0.5 | 0.2 |
| Niacin (B3), mg | 6.6 | 2.6 |
| Vitamin B6, mg | 0.5 | 0.2 |
| Folic Acid, mcg | 186.0 | 74.4 |
| Vitamin B12, mcg | 1.0 | 0.4 |
| Pantothenic Acid (B5), mg | 2.4 | 1.0 |
| Biotin, mcg | 10.6 | 4.2 |
| Choline, mg | 209.0 | 83.6 |
| Calcium, mg | 677.0 | 270.8 |
| Chromium, mcg | 17.3 | 6.9 |
| Copper, mg | 0.2 | 0.1 |
| Iodine, mcg | 76.0 | 30.4 |
| Iron, mg | 8.0 | 3.2 |
| Magnesium, mg | 143.0 | 57.2 |
| Manganese, mg | 1.0 | 0.4 |
| Molybdenum, mcg | 20.0 | 8.0 |
| Phosphorus, mg | 664.0 | 265.6 |
| Selenium, mcg | 26.6 | 10.6 |
| Zinc, mg | 5.0 | 2.0 |
| Potassium, mg | 573.0 | 229.2 |
| Sodium, mg | 287.0 | 114.8 |
| Chloride, mg | 358.0 | 143.2 |
| Inositol, mg | 0.0 | 0.0 |
| L-alanine, mg | 4340.0 | 1736.0 |
| L-arginine, mg | 1900.0 | 760.0 |
| L-asparagine, mg | 0.0 | 0.0 |
| L-aspartate, mg | 1110.0 | 444.0 |
| L-carnitine, mg | 1130.0 | 452.0 |
| L-cystine, mg | 161.9 | 64.8 |
| L-glutamate, mg | 0.0 | 0.0 |
| L-glycine, mg | 2180.0 | 872.0 |
| L-histidine, mg | 600.0 | 240.0 |
| L-isoleucine, mg | 630.0 | 252.0 |
| L-leucine, mg | 0.0 | 0.0 |
| L-lysine, mg | 1440.0 | 576.0 |
| L-methionine, mg | 431.0 | 172.4 |
| L-phenylalanine, mg | 1230.0 | 492.0 |
| L-proline, mg | 2150.0 | 860.0 |
| L-serine, mg | 1160.0 | 464.0 |
| L-taurine, mg | 43.0 | 17.2 |
| L-threonine, mg | 1050.0 | 420.0 |
| L-tryptophan, mg | 229.0 | 91.6 |
| L-tyrosine, mg | 994.6 | 397.8 |
| L-valine, mg | 717.0 | 286.8 |

FIG. 5 shows a non-limiting embodiment of a product label for a nutritional composition for dietary management of Phenylketonuria (PKU).

SKU 37005

NET WEIGHT 2 GAL (7.5 L)

SERVING SIZE 8.5 fl oz (250 mL)

SERVINGS PER PACKAGE 30

REIMBURSEMENT CODE 24359-0705-03 (for USA only)

### MEDICAL FOOD PRODUCT

For the dietary management of Phenylketonuria (PKU). Dispensed by prescription. Phenactin AA Plus is a ready-to-drink metabolic formula product for PKU patients, over 1 year of age. Phenactin Plus contains an advanced fortification blend. Product comes in a 250 mL carton.
PRECAUTIONS Give only to adults and children who are under medical supervision for proven PKU. Protein in prescribed amounts must be supplemented to completely meet phenylalanine requirements.

### NOT FOR PARENTERAL USE•USE AS DIRECTED BY PHYSICIAN

### INGREDIENTS

Water, amino acid blend (L-leucine, L-tyrosine, L-lysine HCl, L-valine, L-aspartic, L-proline, L-threonine, L-isoleucine, L-serine, L-arginine, L-histidine, L-cystine, glycine, L-methionine, L-tryptophan, L-alanine, taurine, L-carnitine, maltodextrin), vitamins and minerals (calcium lactate, monosodium phosphate, monopotassium phosphate, choline bitartrate, magnesium amino acid chelate, sodium ascorbate, ascorbic acid, dl-alpha-tocopheryl acetate, Fe amino acid chelate, niacinamide, zinc amino acid chelate, calcium d-pantothenate, manganese sulfate, riboflavin, B6 pyridoxine HCl, thiamin HCl, copper gluconate, folic acid, vitamin A palmitate, potassium iodide, sodium selenite, cholecalciferol, K1 phytonadione, K2 MK-7, sodium molybdate, cholecalciferol, chromium chloride, biotin, B12 cyanocobalamin), canola oil, sugar, natural flavors (propylene glycol, water, potassium sorbate), inulin, cellulose gel and carboxymethylcellulose sodium, food starch modified, sodium hexametaphosphate, DHA algal oil, acesulfame potassium, carrageenan, sucralose. Contains corn and soy.

### PREPARATION

Ready to drink. Shake well.

### STORAGE

Store in cool, dry place. Refrigerate after opening. Do not freeze.

### TO REQUEST A SAMPLE

Call 866 456 9776, opt 2 or complete form: www.samples.cambrooke.com

### PHENACTIN AA PLUS

| Nutrients | 8.5 fl oz (250 mL) | Per 100 mL |
|---|---|---|
| Calories | 186 | 74 |
| Calories From Fat | 57 | 23 |
| Protein Equivalent, g | 20 | 8 |
| Free Amino Acids, g | 23 | 9 |
| Carbohydrates, g | 13 | 5 |
| Sugar, g | 5 | 2 |
| Sugar Alcohols, g | 0 | 0 |
| Dietary Fiber, g | 2.8 | 1 |
| Fat, g | 6 | 2.4 |
| Saturated Fat, g | 0.5 | 0.2 |
| Trans Fat, g | 0.0 | 0.0 |
| DHA, mg | 150.0 | 60.0 |
| Cholesterol, mg | 0.4 | 0 |
| Vitamin A, IU | 1100.0 | 440.0 |
| Vitamin C, mg | 40.0 | 16.0 |
| Vitamin D, IU | 620.0 | 248.0 |
| Vitamin E, IU | 10.0 | 4.0 |
| Vitamin K1, mcg | 20.0 | 8.0 |
| Vitamin K2 (MK-7), mcg | 20.0 | 8.0 |
| Thiamin (B1), mg | 0.5 | 0.2 |
| Riboflavin (B2), mg | 0.5 | 0.2 |
| Niacin (B3), mg | 6.6 | 2.6 |
| Vitamin B6, mg | 0.5 | 0.2 |
| Folic Acid, mcg | 186.0 | 74.4 |
| Vitamin B12, mcg | 1.0 | 0.4 |
| Pantothenic Acid (B5), mg | 2.4 | 1.0 |
| Biotin, mcg | 10.6 | 4.2 |
| Choline, mg | 209.0 | 83.6 |
| Calcium, mg | 677.0 | 270.8 |
| Chromium, mcg | 17.3 | 6.9 |
| Copper, mg | 0.2 | 0.1 |
| Iodine, mcg | 76.0 | 30.4 |
| Iron, mg | 8.0 | 3.2 |
| Magnesium, mg | 143.0 | 57.2 |
| Manganese, mg | 1.0 | 0.4 |
| Molybdenum, mcg | 20.0 | 8.0 |
| Phosphorus, mg | 664.0 | 265.6 |
| Selenium, mcg | 26.6 | 10.6 |
| Zinc, mg | 5.0 | 2.0 |
| Potassium, mg | 573.0 | 229.2 |
| Sodium, mg | 287.0 | 114.8 |
| Chloride, mg | 350.0 | 140.0 |
| Inositol, mg | 0.0 | 0.0 |
| L-alanine, mg | 423.0 | 169.2 |
| L-arginine, mg | 951.0 | 380.4 |
| L-asparagine, mg | 0.0 | 0.0 |
| L-aspartate, mg | 1760.0 | 704.0 |
| L-carnitine, mg | 18.0 | 7.2 |
| L-cystine, mg | 245.4 | 98.2 |
| L-glutamate, mg | 0.0 | 0.0 |
| L-glycine, mg | 689.0 | 275.6 |
| L-histidine, mg | 882.0 | 352.8 |
| L-isoleucine, mg | 1570.0 | 628.0 |
| L-leucine, mg | 4700.0 | 1880.0 |
| L-lysine, mg | 1830.0 | 732.0 |
| L-methionine, mg | 570.0 | 228.0 |
| L-phenylalanine, mg | 0.0 | 0.0 |
| L-proline, mg | 1700.0 | 680.0 |
| L-serine, mg | 1220.0 | 488.0 |
| L-taurine, mg | 75.0 | 30.0 |
| L-threonine, mg | 1590.0 | 636.0 |
| L-tryptophan, mg | 480.0 | 192.0 |
| L-tyrosine, mg | 1549.7 | 619.9 |
| L-valine, mg | 1800.0 | 720.0 |

FIG. 6 shows a non-limiting embodiment of a product label for a nutritional composition for dietary management of Methylmalonic Acidemia and Propionic Acidemia (MMA/PA).

SKU 37003

NET WEIGHT 2 GAL (7.5 L)

SERVING SIZE 8.5 fl oz (250 mL)

SERVINGS PER PACKAGE 30

REIMBURSEMENT CODE 24359-0703-03 (for USA only)

### MEDICAL FOOD PRODUCT

For the dietary management of Methylmalonic Acidemia and Propionic Acidemia (MMA/PA). Dispensed by prescription. Promactin AA Plus is a ready-to-drink metabolic formula product for Propionic Acidemia and Methylmalonic Acidemia patients, over 1 year of age. Promactin contains an advanced fortification blend. Product comes in a 250 mL carton.

PRECAUTIONS For the dietary management of Methylmalonic Acidemia (MMA) and Propionic Acidemia (PA). Use as directed by physician. Must be administered under medical supervision only.

### NOT FOR PARENTERAL USE•USE AS DIRECTED BY PHYSICIAN

### INGREDIENTS

Water, amino acid blend (L-leucine, L-aspartic, L-lysine HCl, L-alanine, L-arginine, L-serine, L-histidine, L-tyrosine, L-phenylalanine, L-proline, L-glysine, L-cystine, L-tryptophan, L-isoleucine, taurine, L-carnitine, maltodextrin), vitamins and minerals (calcium lactate, monosodium phosphate, monopotassium phosphate, choline bitartrate, magnesium amino acid chelate, sodium ascorbate, ascorbic acid, dl-alpha-tocopheryl acetate, Fe amino acid chelate, niacinamide, zinc amino acid chelate, calcium d-pantothenate, manganese sulfate, riboflavin, B6 pyridoxine HCl, thiamin HCl, copper gluconate, folic acid, vitamin A palmitate, potassium iodide, sodium selenite, cholecalciferol, K1 phytonadione, K2 MK-7, sodium molybdate, cholecalciferol, chromium chloride, biotin, B12 cyanocobalamin), canola oil, sugar, natural flavors (propylene glycol, water, potassium sorbate), cellulose gel and carboxymethylcellulose sodium, food starch modified, sodium hexametaphosphate, DHA algal oil, acesulfame potassium, carrageenan, sucralose. Contains corn and soy.

### PREPARATION

Ready to drink. Shake well.

### STORAGE

Store in cool, dry place. Refrigerate after opening. Do not freeze.

### TO REQUEST A SAMPLE

Call 866 456 9776, opt 2 or complete form: www.samples.cambrooke.com

### PROMACTIN AA PLUS

| Nutrients | 8.5 fl oz (250 mL) | Per 100 mL |
|---|---|---|
| Calories | 183 | 73 |
| Calories From Fat | 57 | 23 |
| Protein Equivalent, g | 20 | 8 |
| Free Amino Acids, g | 23 | 9 |
| Carbohydrates, g | 12 | 5 |
| Sugar, g | 5 | 2 |
| Sugar Alcohols, g | 0 | 0 |
| Dietary Fiber, g | 1 | 0 |
| Fat, g | 6.0 | 2.4 |
| Saturated Fat, g | 0.5 | 0.2 |
| Trans Fat, g | 0.0 | 0.0 |
| DHA, mg | 150.0 | 60.0 |
| Cholesterol, mg | 0.4 | 0 |
| Vitamin A, IU | 1100.0 | 440.0 |
| Vitamin C, mg | 40.0 | 16.0 |
| Vitamin D, IU | 620.0 | 248.0 |
| Vitamin E, IU | 10.0 | 4.0 |
| Vitamin K1, mcg | 20.0 | 8.0 |
| Vitamin K2 (MK-7), mcg | 20.0 | 8.0 |
| Thiamin (B1), mg | 0.5 | 0.2 |
| Riboflavin (B2), mg | 0.5 | 0.2 |
| Niacin (B3), mg | 6.6 | 2.6 |
| Vitamin B6, mg | 0.5 | 0.2 |
| Folic Acid, mcg | 186.0 | 74.4 |
| Vitamin B12, mcg | 1.0 | 0.4 |
| Pantothenic Acid (B5), mg | 2.4 | 1.0 |
| Biotin, mcg | 10.6 | 4.2 |
| Choline, mg | 209.0 | 83.6 |
| Calcium, mg | 700.0 | 280.0 |
| Chromium, mcg | 17.3 | 6.9 |
| Copper, mg | 0.2 | 0.1 |
| Iodine, mcg | 76.0 | 30.4 |
| Iron, mg | 8.0 | 3.2 |
| Magnesium, mg | 143.0 | 57.2 |
| Manganese, mg | 1.0 | 0.4 |
| Molybdenum, mcg | 20.0 | 8.0 |
| Phosphorus, mg | 664.0 | 265.6 |
| Selenium, mcg | 26.6 | 10.6 |
| Zinc, mg | 5.0 | 2.0 |
| Potassium, mg | 573.0 | 229.2 |
| Sodium, mg | 287.0 | 114.8 |
| Chloride, mg | 607.0 | 242.8 |
| Inositol, mg | 0.0 | 0.0 |
| L-alanine, mg | 2270.0 | 908.0 |
| L-arginine, mg | 2178.0 | 871.2 |
| L-asparagine, mg | 0.0 | 0.0 |
| L-aspartate, mg | 2900.0 | 1160.0 |
| L-carnitine, mg | 20.0 | 8.0 |
| L-cystine, mg | 277.6 | 111.0 |
| L-glutamate, mg | 0.0 | 0.0 |
| L-glycine, mg | 107.0 | 42.8 |
| L-histidine, mg | 1450.0 | 580.0 |
| L-isoleucine, mg | 68.0 | 27.2 |
| L-leucine, mg | 3130.0 | 1252.0 |
| L-lysine, mg | 2280.0 | 912.0 |
| L-methionine, mg | 0.0 | 0.0 |
| L-phenylalanine, mg | 1420.0 | 568.0 |
| L-proline, mg | 1020.0 | 408.0 |
| L-serine, mg | 1480.0 | 592.0 |
| L-taurine, mg | 57.0 | 22.8 |
| L-threonine, mg | 0.0 | 0.0 |
| L-tryptophan, mg | 613.0 | 245.2 |
| L-tyrosine, mg | 971.5 | 388.6 |
| L-valine, mg | 0.0 | 0.0 |

FIG. 7 shows a non-limiting embodiment of a product label for a nutritional composition for dietary management of Maple Syrup Urine Disease (MSUD).

SKU 37004

NET WEIGHT 2 GAL (7.5 L)

SERVING SIZE 8.5 fl oz (250 mL)

SERVINGS PER PACKAGE 30

REIMBURSEMENT CODE 24359-0704-03 (for USA only)

### MEDICAL FOOD PRODUCT

For the dietary management of Maple Syrup Urine Disease (MSUD). Dispensed by prescription. Vilactin AA Plus is a ready-to-drink metabolic formula product for MSUD patients, over 1 year of age. Vilactin AA Plus contains an advanced fortification blend. Comes in a 250 mL carton.
PRECAUTIONS For the dietary management of Maple Syrup Urine Disease (MSUD). Use as directed by physician. Must be administered under medical supervision only.

### NOT FOR PARENTERAL USE•USE AS DIRECTED BY PHYSICIAN

### INGREDIENTS

Water, amino acid blend (L-glutamine, L-alanine, L-lysine HCl, L-phenylalanine, L-tyrosine, L-threonine, L-histidine, L-proline, asparagine, L-tryptophan, L-arginine, L-serine, L-glutamic acid, glycine, L-cystine, L-methionine, taurine, L-carnitine), vitamins and minerals (calcium lactate, monosodium phosphate, monopotassium phosphate, choline bitartrate, magnesium amino acid chelate, sodium ascorbate, ascorbic acid, dl-alpha-tocopheryl acetate, iron amino acid chelate, niacinamide, zinc amino acid chelate, calcium d-pantothenate, manganese sulfate, riboflavin, B6 Pyridoxal Phosphate, thiamin HCl, copper gluconate, L-methylfolate, vitamin A palmitate, potassium iodide, sodium selenite, cholecalciferol, K1 phytonadione, K2 MK-7, sodium molybdate, cholecalciferol, chromium chloride, biotin, B12 methylcobalamin), canola oil, sugar, natural flavors (propylene glycol, water, potassium sorbate), inulin, cellulose gel and carboxymethylcellulose sodium, food starch modified, sodium hexametaphosphate, DHA algal oil, acesulfame potassium, carrageenan, sucralose. Contains corn and soy.

### PREPARATION

Ready to drink. Shake well.

### STORAGE

Store in cool, dry place. Refrigerate after opening. Do not freeze.

### TO REQUEST A SAMPLE

Call 866 456 9776, opt 2 or complete form: www.samples.cambrooke.com

### VILACTIN AA PLUS

| Nutrients | 8.5 fl oz (250 mL) | Per 100 mL |
|---|---|---|
| Calories | 183 | 73 |
| Calories From Fat | 57 | 23 |
| Protein Equivalent, g | 20 | 8 |
| Free Amino Acids, g | 23 | 9 |
| Carbohydrates, g | 13 | 5 |
| Sugar, g | 5 | 2 |
| Sugar Alcohols, g | 0 | 0 |
| Dietary Fiber, g | 3 | 1 |
| Fat, g | 6.0 | 2.4 |
| Saturated Fat, g | 0.3 | 0.1 |
| Trans Fat, g | 0.0 | 0.0 |
| DHA, mg | 150.0 | 60.0 |
| Cholesterol, mg | 0 | 0 |
| Vitamin A, IU | 1100.0 | 440.0 |
| Vitamin C, mg | 40.0 | 16.0 |
| Vitamin D, IU | 620.0 | 248.0 |
| Vitamin E, IU | 10.0 | 4.0 |
| Vitamin K1, mcg | 20.0 | 8.0 |
| Vitamin K2 (MK-7), mcg | 20.0 | 8.0 |
| Thiamin (B1), mg | 0.5 | 0.2 |
| Riboflavin (B2), mg | 0.5 | 0.2 |
| Niacin (B3), mg | 6.6 | 2.6 |
| Vitamin B6, mg | 0.5 | 0.2 |
| Folic Acid, mcg | 186.0 | 74.4 |
| Vitamin B12, mcg | 1.0 | 0.4 |
| Pantothenic Acid (B5), mg | 2.4 | 1.0 |
| Biotin, mcg | 10.6 | 4.2 |
| Choline, mg | 98.3 | 39.3 |
| Calcium, mg | 677.0 | 270.8 |
| Chromium, mcg | 17.4 | 7.0 |
| Copper, mg | 0.2 | 0.1 |
| Iodine, mcg | 76.0 | 30.4 |
| Iron, mg | 8.0 | 3.2 |
| Magnesium, mg | 143.0 | 57.2 |
| Manganese, mg | 1.0 | 0.4 |
| Molybdenum, mcg | 20.0 | 8.0 |
| Phosphorus, mg | 664.0 | 265.6 |
| Selenium, mcg | 26.6 | 10.6 |
| Zinc, mg | 5.0 | 2.0 |
| Potassium, mg | 573.0 | 229.2 |
| Sodium, mg | 287.0 | 114.8 |
| Chloride, mg | 350.0 | 140.0 |
| Inositol, mg | 0.0 | 0.0 |
| L-alanine, mg | 2591.0 | 1036.4 |
| L-arginine, mg | 2085.0 | 834.0 |
| L-asparagine, mg | 1477.0 | 590.8 |
| L-aspartate, mg | 0.0 | 0.0 |
| L-carnitine, mg | 40.0 | 16.0 |
| L-cystine, mg | 565.0 | 226.0 |
| L-glutamate, mg | 0.0 | 0.0 |
| L-glycine, mg | 1416.0 | 566.4 |
| L-histidine, mg | 1475.0 | 590.0 |
| L-isoleucine, mg | 0.0 | 0.0 |
| L-leucine, mg | 0.0 | 0.0 |
| L-lysine, mg | 1808.0 | 723.2 |
| L-methionine, mg | 677.0 | 270.8 |
| L-phenylalanine, mg | 1533.0 | 613.2 |
| L-proline, mg | 2551.0 | 1020.4 |
| L-serine, mg | 1607.0 | 642.8 |
| L-taurine, mg | 748.0 | 299.2 |
| L-threonine, mg | 1734.0 | 693.6 |
| L-tryptophan, mg | 1614.0 | 645.6 |
| L-tyrosine, mg | 1658.0 | 663.2 |
| L-valine, mg | 0.0 | 0.0 |

### DETAILED DESCRIPTION OF INVENTION

The disclosure relates to compositions for dietary management of inborn errors of metabolism. Compositions for support bone health of subjects having inborn errors of metabolism, provide a source of complete nutrition to subjects having inborn errors of metabolism, or support bone health and provide source of complete nutrition to subjects having inborn errors of metabolism. The term “nutritionally complete” refers to a composition having a complete nutritional profile of macro and micronutrients (e.g., containing carbohydrates, protein, fats, vitamins and minerals) for subjects 1 year of age and older.

### Inborn Errors of Metabolism

In some aspects, the disclosure relates to compositions for dietary management of inborn errors of metabolism. As used herein the term “inborn error of metabolism” refers to a disease or disorder characterized by defective function of a metabolic enzyme. Generally, an inborn error of metabolism is inherited (e.g., a congenital disease or disorder). Inborn errors of metabolism can be divided into several types, including disorders of carbohydrate metabolism (e.g., glycogen storage disease), disorders of amino acid metabolism, urea cycle disorders (e.g., carbamoyl phosphate synthetase I deficiency), disorders of organic acid metabolism (e.g., alcaptonuria), disorders of mitochondrial metabolism (e.g., Leigh syndrome), disorders of fatty acid oxidation (e.g., Medium-chain acyl-coenzyme A dehydrogenase deficiency), and lysosomal storage disorders (e.g., Gaucher's disease).

In some embodiments, compositions described by the disclosure are for dietary management of disorders of amino acid metabolism, which are characterized by the inability of a subject to metabolize a particular amino acid. Examples of disorders of amino acid metabolism and the gene(s) associated with each disease include phenylketonuria (PKU; phenylalanine hydroxylase), Maple Syrup Urine Disease (MSUD; branched-chain alpha-keto acid dehydrogenase complex), Homocystinuria (HCU; cystathionine beta synthase), Isovaleric acidaemia (IVA; isovaleric acid-CoA dehydrogenase), and Methylmalonic acidaemia/Propionic acidaemia (MMA/PA; methylmalonyl-CoA mutase/propionyl-CoA carboxylase).

### Nutritional Formulas for Dietary Management of Inborn Errors of Metabolism

In some aspects, the disclosure provides a nutritional composition for dietary management of an inborn error of metabolism comprising: a protein source, a fat source, a carbohydrate source, and a bone health composition, wherein the protein source comprises at least six free amino acids. In some embodiments, the nutritional composition is nutritionally complete.

The protein source is a mixture of free amino acids. As used herein, the term “free amino acid” refers to a single amino acid that does not require digestion or metabolism. Generally, a free amino acid is to the L-isomer of the amino acid (e.g., L-alanine) as opposed to the D-isomer of the amino acid (e.g., D-alanine). However, in some embodiments, a free amino acid is the D-isomer of the amino acid.

In some embodiments, a protein source comprises a mixture of free amino acids (e.g., a blend of amino acids). Such a mixture of amino acid comprises essential amino acids and non-essential amino acids. The essential amino acids are histidine, isoleucine, leucine, lysine, methionine, phenylalanine, threonine, tryptophan, and valine. The amino acids present in a particular amino acid mixture of a protein source will be determined by the inborn error of metabolism to be managed by administration of the nutritional composition. For example, a composition for the management of phenylketonuria will have, at most, a minimal amount of phenylalanine. In some embodiments, a protein source (e.g., a blend of amino acids) comprises at least six free amino acids. In some embodiments, the at least six free amino acids are selected from the group consisting of: histidine, isoleucine, leucine, lysine, methionine, phenylalanine, threonine, tryptophan, and valine. Examples of protein source blends are shown in Table 3, Table 4, Table 6, Table 8, Table 9, and Table 10.

Amino acid content affects the pH of nutritional compositions. In some embodiments, the pH of a nutritional composition described by the disclosure ranges from about pH 3.0 to about pH 7.0. In some embodiments, the pH of a nutritional composition described by the disclosure ranges from about pH 4.0 to about pH 6.0. In some embodiments, the pH of a nutritional composition described by the disclosure ranges from about pH 5.0 to about pH 6.0. In some embodiments, the pH of a nutritional composition described by the disclosure is about pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, or pH 6.0. In some embodiments, the pH of a nutritional composition is less than pH 5.0.

The nutritional compositions as described herein comprise a fat source, which can be a single type of fat, or a combination of more than one type of fat. A fat source may comprise saturated fats, unsaturated fats or a combination of saturated fats and unsaturated fats. Fat can comprise long chain fatty acids, short chain fatty acids or a combination of long chain fatty acids and short chain fatty acids. In some embodiments, fat comprises triglycerides, such as short-chain triglycerides, long-chain triglycerides, medium-chain triglycerides or a combination of two or three of the foregoing (e.g., short-chain triglycerides, long-chain triglycerides and medium-chain triglycerides; short-chain triglycerides and long-chain triglycerides; short-chain triglycerides and medium-chain triglycerides; medium-chain triglycerides and long-chain triglycerides). Examples of fat sources include but are not limited to: butter, animal fat (for example beef or chicken fat), vegetable oil (for example avocado, corn, and soybean), olive oil, canola oil, coconut oil, cocoa butter, fish oil, nuts (for example macadamia and peanut) and nut oils.

In some embodiments, the fat source is between about 1.8% and about 2.8% of the total weight of the composition. In some embodiments, the fat source is about 1.8%, about 1.9%, about 2.0%, about 2.1%, about 2.2%, about 2.3%, about 2.4%, about 2.5%, about 2.6%, about 2.7%, or about 2.8% of the total weight of the composition. In some embodiments, the fat source is at a concentration of between about 19 g/L and about 29 g/L.

Emulsifiers can be used to improve stability and texture of nutritional compositions described herein. In some aspects, the nutritional compositions comprise an emulsifier or emulsifying agent (e.g., a surfactant), such as sodium stearoyl lactylate (SSL), lecithin, starches, gums and biopolymeric emulsifiers. In some embodiments, the emulsifier is a modified starch, such as octenyl succinate starch (e.g., OSA starch) or other modified starch, that interferes with interaction of fat and protein source in the formula, such as by binding of the octenyl moiety of the starch to fat globules.

Nutritional compositions comprise a carbohydrate source, such as starch, gum and/or fiber. In some embodiments, carbohydrate acts as a food stabilizer. A carbohydrate source can be a single type of carbohydrate, or a combination of several types of carbohydrate. In some embodiments, carbohydrate is less than about 2.1% of the total weight of the composition. In some embodiments, carbohydrate is a gum. In some embodiments, carbohydrate comprises carboxymethyl cellulose, carrageenan or carboxymethyl cellulose and carrageenan.

In some embodiments, the composition contains supplemental dietary fiber. Dietary fiber can be a single type of fiber or a combination of more than one type of fiber. In some embodiments, the dietary fibers are soluble fibers, non-soluble fibers, or dietary fibers and non-soluble fibers. In some embodiments, the fiber comprises very low density lipoprotein (vldl)- and low density lipoprotein (ldl)-reducing soluble fibers. In some embodiments, the fiber is one or more selected from the group consisting of inulin, pectin, cellulose gum (carboxymethyl cellulose) and carrageenan.

In some embodiments, nutritional compositions further comprise at least one (one or more) sweetener, such as acesulfame potassium, sucralose, aspartame, lo han guo, stevia, erythritol, xylitol, maltitol, sorbitol, other nutritive or non-nutritive sources. In some embodiments, the composition does not contain artificial or nutritive sweeteners.

The nutritional compositions described herein comprise at least one vitamin and at least one mineral. In some embodiments, the at least one vitamin is selected from the group consisting of Vitamin A, Vitamin D, Vitamin E, Biotin, folic acid, niacin, pantothenic acid, Vitamin B1, Vitamin B12, Vitamin B2, Vitamin B6, Vitamin C, and Vitamin K. Examples of vitamin sources include: palmitates, beta-carotene, ergocalciferol, cholecalciferol, dl-alpha tocopheryl acetate, d-alpha tocopheryl acetate, dl-alpha tocotrienols, calcium panthothenate, pantothenol, pantothenic acid, cyanocobalamin, methylcobalamin, sodium ascorbate, calcium ascorbate, ascorbic acid, pyridoxine hydrochloride, pyridoxal 5′phosphate, riboflavin, thiamin, folic acid, phylloquinone, phytomenadione, phytonadione, menaquinones (e.g., MK-4 and MK-7) and biotin. Any combination of vitamins can be included in the nutritional compositions. In some embodiments, a nutritional composition comprises at least one vitamin in an amount listed in Table 2 or Table 7. In some embodiments, a nutritional composition comprises all of the vitamins in Table 2 or Table 7, such as the vitamins listed in the quantities listed in Table 2 or Table 7.

In some embodiments, the nutritional composition comprises at least one mineral selected from the group consisting of: calcium, chromium, copper, iodine, iron, magnesium, manganese, molybdenum, phosphorus, potassium, selenium, sodium, and zinc. Examples of mineral sources include calcium lactate, calcium gluconate, calcium pantothenate, calcium lactate gluconate, calcium phosphate, calcium carbonate, calcium citrate, calcium phosphate, magnesium phosphate, potassium phosphate, choline chloride, phosphatidylcholine, choline bitartrate, lecithin, magnesium chloride, magnesium oxide, magnesium gluconate, magnesium phosphate, magnesium malate, magnesium citrate, magnesium fumarate, inositol hexanicotinate, nicotinamide, niacinamine, zinc carbonate, zinc citrate, zinc sulfate, zinc gluconate, zinc bisglycinate, zinc fumarate, manganese chloride, manganese gluconate, manganese sulfate, manganese picolinate, monosodium sulfate, monopotassium phosphate, iron sulfate, iron citrate, iron gluconate, ferrous fumarate, cupric oxide, copper gluconate, copper sulfate, copper carbonate, chromium picolinate, chromium chloride, chromium polynicotinate, chromium chloride, sodium chloride, potassium chloride, magnesium chloride, manganese chloride, choline chloride, potassium chloride, potassium citrate, potassium iodide, potassium sodium tartrate, potassium bisulfite, potassium iodide, sodium selenite, selenomethionine, sodium selenite, potassium molybdate, sodium molybdate, sodium chloride, sodium citrate, or amino acid chelates (e.g., zinc chelate, magnesium chelate) of any of the foregoing. Any combination of minerals can be included in the nutritional compositions. In some embodiments, a nutritional composition comprises at least one mineral in an amount listed in Table 2 or Table 7. In some embodiments, a nutritional composition comprises all of the minerals listed in Table 2 or Table 7, such as the minerals in the quantities listed in Table 2 or Table 7.

The addition of certain minerals, such as zinc, copper and/or iron, to food products is known to contribute to fat rancidity in food products. Chelation of sensitive minerals may therefore reduce fat rancidity by preventing minerals associated with fat rancidity from interacting with fats that are present in a composition. In some embodiments, the nutritional compositions comprise a chelating agent, such as phosphates or phosphonates, EDTA and sodium hexametaphosphate (SHMP). In some embodiments, the chelating agent is sodium hexametaphosphate (SHMP). Other methods of reducing fat rancidity, such as the addition of antioxidants, are also known in the art. In some embodiments, nutritional compositions described herein comprise at least one antioxidant, such as ascorbate, ascorbic acid (Vitamin C), cysteine and tocopherols.

Several embodiments of a nutritionally complete nutritional composition for dietary management of an inborn error of metabolism are shown in Table 5. Table 5 provides ranges of each component of a nutritional composition. In some embodiments, the amino acid blend of the composition is selected from the amino acid blends listed in Table 4 or Table 10. The amino acid blend of the composition is determined by the inborn error of metabolism to be managed by the composition. For example, if the condition to be managed is MSUD, Blend #2 from Table 4 or Blend #2 from Table 10 can be used. In some embodiments, the vitamin and mineral blend referred to Table 5 comprises the vitamin and mineral blend represented in Table 2 or Table 7.

### Compositions Supporting Bone Health

In some aspects, the disclosure relates to compositions that support bone health in subjects having inborn errors of metabolism. Subjects having certain types of inborn errors of metabolism have increased risk of bone abnormalities (e.g., reduced bone mineral density). Accordingly in some embodiments, the disclosure provides compositions that support the bone health of subjects having inborn errors of metabolism. Bone health compositions can be solid (e.g., a powder) or liquid.

Bone health compositions described herein comprise vitamins and minerals. In some embodiments, bone health compositions comprise Vitamin D, calcium, phosphorus, zinc, magnesium, and Vitamin K. For example, the amount of each component in about 1.5 g (without inulin) to about 4.0 g (with inulin) of bone health composition described by the disclosure is shown in Table 1. The amount of bone health composition included in a serving of nutritional composition ranges from about 0.5 g to about 5.0 g. In some embodiments, the amount of bone health composition included in a serving of nutritional composition ranges from about 1.0 g to about 4.0 g. In some embodiments, the amount of bone health composition included in a serving of nutritional composition ranges from about 1.5 g to about 3.5 g. In some embodiments, the amount of bone health composition included in a serving of nutritional composition ranges from about 2.0 g to about 3.0 g. In some embodiments, the amount of bone health composition included in a serving of nutritional composition is about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, about 3.2 g, about 3.3 g, about 3.4 g, about 3.5 g, about 3.6 g, about 3.7 g, about 3.8 g, about 3.9 g, or about 4.0 g.

In some embodiments, bone health compositions are added to supplement nutritional formulas and compositions for dietary management of inborn errors of metabolism. For example, a bone health composition may be combined with a protein source. In some embodiments, the protein source comprises one or more amino acids selected from the group consisting of: histidine, isoleucine, leucine, lysine, methionine, phenylalanine, threonine, tryptophan, and valine. In some embodiments, the protein source comprises at least six amino acids selected from the group consisting of: histidine, isoleucine, leucine, lysine, methionine, phenylalanine, threonine, tryptophan, and valine.

TABLE 1
Ranges of Components in Bone Health Compositions

| | Minimum | Maximum |
|---|---|---|
| Vitamin/Mineral | Amount | Amount |
| vitamin D3 (IU) | 500 | 770 |
| calcium (mg) | 550 | 840 |
| phosphorus (mg) | 475 | 720 |
| zinc (mg) | 4 | 6 |
| magnesium (mg) | 125 | 195 |
| vitamin K1 (mcg) | 10 | 25 |
| vitamin K2 (mcg) | 10 | 25 |
| inulin (g) | 0 | 2.5 |

TABLE 2
Examples of Ranges of Vitamins and Minerals in
Nutritional Compositions

| | Minimum | Maximum |
|---|---|---|
| Vitamin/Mineral | Amount | Amount |
| Vitamin A (IU) | 950 | 1450 |
| Vitamin D3 (IU) | 500 | 770 |
| Vitamin E (IU) | 7 | 12 |
| Biotin (μg) | 8 | 13 |
| Folic Acid (mg) | 0.14 | 0.23 |
| Niacin (mg) | 5.25 | 7.95 |
| Pantothenic Acid (mg) | 1.9 | 2.9 |
| Vitamin B1 (mg) | 0.43 | 0.65 |
| Vitamin B12 (μg) | 0.75 | 1.2 |
| Vitamin B2 (mg) | 0.43 | 0.65 |
| Vitamin B6 (mg) | 0.43 | 0.65 |
| Vitamin C (mg) | 35 | 55 |
| Vitamin K1 (μg) | 10 | 25 |
| Vitamin K2 (μg) | 10 | 25 |
| Calcium (mg) | 550 | 840 |
| Chromium (μg) | 13.5 | 20.9 |
| Copper (mg) | 0.15 | 0.25 |
| Iodine (μg) | 60 | 91.5 |
| Iron (mg) | 6 | 9.75 |
| Magnesium (mg) | 125 | 195 |
| Manganese (mg) | 0.75 | 1.25 |
| Molybdenum (μg) | 15 | 25 |
| Phosphorus (mg) | 475 | 720 |
| Potassium (mg) | 375 | 570 |
| Selenium (μg) | 21 | 32 |
| Sodium (mg) | 100 | 200 |
| Zinc (mg) | 4 | 6 |
| sodium ascorbate | 0.06 | 0.09 |
| ascorbic acid | 0.03 | 0.05 |

TABLE 3
Examples of Free Amino Acid Ranges in Nutritional Compositions

| AA Blend/Serving | Minimum | Maximum |
|---|---|---|
| (8.5 fl oz.) | mg/serving | mg/serving |
| Glycine (mg) | 85 | 2510 |
| L-Alanine (mg) | 360 | 5000 |
| L-Arginine (mg) | 435 | 2500 |
| L-Asparagine (mg) | 0 | 2070 |
| L-Aspartic Acid (mg) | 0 | 2900 |
| L-Carnitine (mg) | 15 | 1140 |
| L-Cystine (mg) | 190 | 865 |
| L-Glutamic Acid (mg) | 0 | 400 |
| L-Glutamine (mg) | 195 | 2870 |
| L-Histidine (mg) | 510 | 1450 |
| L-Isoleucine (mg) | 0 | 1570 |
| L-Leucine (mg) | 0 | 4710 |
| L-Lysine (mg) | 775 | 2300 |
| L-Methionine (mg) | 0 | 600 |
| L-Phenylalanine (mg) | 0 | 1500 |
| L-Proline (mg) | 500 | 2160 |
| L-Serine (mg) | 380 | 1485 |
| L-Threonine (mg) | 0 | 1600 |
| L-Tryptophan (mg) | 195 | 615 |
| L-Tyrosine (mg) | 375 | 2260 |
| L-Valine (mg) | 0 | 1800 |
| Taurine | 35 | 80 |

TABLE 4
Examples of Amino Acid Blends

| | Blend 1 | | Blend 2 | | Blend 3 | | Blend 4 | | Blend 5 | |
|---|---|---|---|---|---|---|---|---|---|---|
| Amino Acid/ | (PKU) | | (MSUD) | | (HCU) | | (MMA/PA) | | (IVA) | |
| 8 fl oz.serving | MIN | MAX | MIN | MAX | MIN | MAX | MIN | MAX | MIN | MAX |
| Glycine (mg) | 551.2 | 827 | 216 | 324 | 1200 | 1800 | 781.6 | 1172.4 | 1745.6 | 2618 |
| L-Alanine (mg) | 338.4 | 508 | 864 | 1296 | 856 | 1284 | 1816 | 2724 | 3479.2 | 5219 |
| L-Arginine (mg) | 760.8 | 1141 | 408 | 612 | 1240 | 1860 | 1742.4 | 2613.6 | 1522.4 | 2284 |
| L-Asparagine (mg) | 0 | 0 | 464 | 696 | 0 | 0 | 0 | 0 | 0 | 0 |
| L-Aspartic Acid (mg) | 1408.8 | 2113 | 0 | 0 | 1549.6 | 2324 | 2320 | 3480 | 894.4 | 1342 |
| L-Carnitine (mg) | 14.4 | 21.6 | 16 | 24 | 16.8 | 25.2 | 16 | 24 | 910.4 | 1366 |
| L-Cystine (mg) | 621.6 | 932 | 176 | 264 | 648 | 972 | 689.6 | 1034.4 | 178.4 | 268 |
| L-Glutamic Acid (mg) | 0 | 0 | 280 | 420 | 0 | 0 | 0 | 0 | 0 | 0 |
| L-Glutamine (mg) | 184.8 | 277 | 2296 | 3444 | 1025.6 | 1538 | 964 | 1446 | 1496 | 2244 |
| L-Histidine (mg) | 705.6 | 1058 | 568 | 852 | 744 | 1116 | 1162.4 | 1743.6 | 480 | 720 |
| L-Isoleucine (mg) | 1256 | 1884 | 0 | 0 | 1146.4 | 1720 | 54.4 | 81.6 | 509.6 | 764 |
| L-Leucine (mg) | 3765.6 | 5648 | 0 | 0 | 1965.6 | 2948 | 2504 | 3756 | 0 | 0 |
| L-Lysine (mg) | 1468 | 2202 | 728 | 1092 | 1296 | 1944 | 1826.4 | 2739.6 | 1159.2 | 1739 |
| L-Methionine (mg) | 461.6 | 692 | 240 | 360 | 0 | 0 | 0 | 0 | 344.8 | 517 |
| L-Phenylalanine (mg) | 0 | 0 | 704 | 1056 | 864 | 1296 | 1142.4 | 1713.6 | 988.8 | 1483 |
| L-Proline (mg) | 1364.8 | 2047 | 472 | 708 | 1408 | 2112 | 822.4 | 1233.6 | 1725.6 | 2588 |
| L-Serine (mg) | 980.8 | 1471 | 360 | 540 | 1005.6 | 1508 | 1186.4 | 1779.6 | 933.6 | 1400 |
| L-Threonine (mg) | 1276.8 | 1915 | 576 | 864 | 914.4 | 1372 | 0 | 0 | 840 | 1260 |
| L-Tryptophan (mg) | 385.6 | 578 | 424 | 636 | 386.4 | 580 | 490.4 | 735.6 | 183.2 | 275 |
| L-Tyrosine (mg) | 1804.8 | 2707 | 704 | 1056 | 1128 | 1692 | 1142.4 | 1713.6 | 993.6 | 1490 |
| L-Valine (mg) | 1440.8 | 2161 | 0 | 0 | 1250.4 | 1876 | 0 | 0 | 573.6 | 860 |

TABLE 5
Example of a Nutritional Composition

| | Minimum | Maximum | Minimum % | Maximum % |
|---|---|---|---|---|
| Ingredient | g/L | g/L | Total Weight | Total Weight |
| Water | 890 | 750 | 72.47% | 87.5% |
| stabilizer blend | 4 | 6.2 | 0.39% | 0.610% |
| sequestrant | 1 | 1.6 | 0.1% | 0.12% |
| choline | 0 | 1 | 0% | 0.1% |
| carbohydrate blend | 9 | 15 | 0.87% | 1.48% |
| fiber blend | 5 | 10 | 0.48% | 0.98% |
| Emulsifier | 1.9 | 2.9 | 0.18% | 0.29% |
| fat blend | 19 | 29 | 1.84% | 2.85% |
| amino acid blend | 80 | 140 | 7.73% | 13.76% |
| vitamin and | 25 | 40 | 2.42% | 3.93% |
| mineral blend | | | | |
| Sweetener | 0 | 1.5 | 0% | 0.15% |
| Flavor | 0 | 20 | 0% | 1.97% |

TABLE 6
Examples of Amino Acid Blends in Nutritional Compositions

| | AA | Blend 1 | Blend 2 | Blend 3 | Blend 4 | Blend 5 |
|---|---|---|---|---|---|---|
| AA Blend/Serving | Blend/Serving | (PKU) | (MSUD) | (HCU) | (MMA/PA) | (IVA) |
| Glycine (mg) | 689 | 270 | 1500 | 977 | 2182 | 100 |
| L-Alanine (mg) | 423 | 1080 | 1070 | 2270 | 4349 | 1000 |
| L-Arginine (mg) | 951 | 510 | 1550 | 2178 | 1903 | 2000 |
| L-Asparagine (mg) | 0 | 580 | 0 | 0 | 0 | 1800 |
| L-Aspartic Acid (mg) | 1761 | 0 | 1937 | 2900 | 1118 | 1200 |
| L-Carnitine (mg) | 18 | 20 | 21 | 20 | 1138 | |
| L-Cystine (mg) | 777 | 220 | 810 | 862 | 223 | 600 |
| L-Glutamic Acid | 0 | 350 | 0 | 0 | 0 | 400 |
| (mg) | | | | | | |
| L-Glutamine (mg) | 231 | 2870 | 1282 | 1205 | 1870 | 1600 |
| L-Histidine (mg) | 882 | 710 | 930 | 1453 | 600 | 800 |
| L-Isoleucine (mg) | 1570 | 0 | 1433 | 68 | 637 | 0 |
| L-Leucine (mg) | 4707 | 0 | 2457 | 3130 | 0 | 1500 |
| L-Lysine (mg) | 1835 | 910 | 1620 | 2283 | 1449 | 2300 |
| L-Methionine (mg) | 577 | 300 | 0 | 0 | 431 | 600 |
| L-Phenylalanine | 0 | 880 | 1080 | 1428 | 1236 | 1500 |
| (mg) | | | | | | |
| L-Proline (mg) | 1706 | 590 | 1760 | 1028 | 2157 | 600 |
| L-Serine (mg) | 1226 | 450 | 1257 | 1483 | 1167 | 1000 |
| L-Threonine (mg) | 1596 | 720 | 1143 | 0 | 1050 | 500 |
| L-Tryptophan (mg) | 482 | 530 | 483 | 613 | 229 | 480 |
| L-Tyrosine (mg) | 2256 | 880 | 1410 | 1428 | 1242 | 440 |
| L-Valine (mg) | 1801 | 0 | 1563 | 0 | 717 | 500 |
| Taurine | 75 | 60 | 50 | 57 | 43 | 80 |

TABLE 7
Examples of Ranges of Vitamins and Minerals in
Nutritional Compositions

| | Minimum | Maximum |
|---|---|---|
| Vitamin/Mineral | Amount | Amount |
| Vitamin A (IU) | 950 | 1450 |
| Vitamin D3 (IU) | 500 | 770 |
| Vitamin E (IU) | 7 | 12 |
| Biotin (μg) | 8 | 13 |
| Folic Acid (mg) | 0.14 | 0.23 |
| Niacin (mg) | 5.25 | 7.95 |
| Pantothenic Acid (mg) | 1.9 | 2.9 |
| Vitamin B1 (mg) | 0.43 | 0.65 |
| Vitamin B12 (μg) | 0.75 | 1.2 |
| Vitamin B2 (mg) | 0.43 | 0.65 |
| Vitamin B6 (mg) | 0.43 | 0.65 |
| Vitamin C (mg) | 35 | 55 |
| Vitamin K1 (μg) | 10 | 25 |
| Vitamin K2 (μg) | 10 | 25 |
| Calcium (mg) | 550 | 840 |
| Chromium (μg) | 13.5 | 20.9 |
| Copper (mg) | 0.15 | 0.25 |
| Iodine (μg) | 60 | 91.5 |
| Iron (mg) | 6 | 9.75 |
| Magnesium (mg) | 125 | 195 |
| Manganese (mg) | 0.75 | 1.25 |
| Molybdenum (μg) | 15 | 25 |
| Phosphorus (mg) | 475 | 720 |
| Potassium (mg) | 375 | 600 |
| Selenium (μg) | 21 | 32 |
| Sodium (mg) | 100 | 300 |
| Chloride (mg) | 10 | 700 |
| Zinc (mg) | 4 | 6 |
| sodium ascorbate | 0.06 | 0.09 |
| ascorbic acid | 0.03 | 0.05 |

TABLE 8
Examples of Free Amino Acid Ranges in Nutritional Compositions

| | Min | Max |
|---|---|---|
| AA/Serving (8.5 fl oz.) | mg/serving | mg/serving |
| Glycine (mg) | 85.6 | 2616 |
| L-Alanine (mg) | 338.4 | 5208 |
| L-Arginine (mg) | 760.8 | 2613.6 |
| L-Asparagine (mg) | 0 | 1772.4 |
| L-Aspartic Acid (mg) | 0 | 3480 |
| L-Carnitine (mg) | 14.4 | 1236 |
| L-Cystine (mg) | 129.52 | 678 |
| L-Glutamic Acid (mg) | 0 | 0 |
| L-Histidine (mg) | 480 | 1770 |
| L-Isoleucine (mg) | 0 | 1884 |
| L-Leucine (mg) | 0 | 5640 |
| L-Lysine (mg) | 1152 | 2736 |
| L-Methionine (mg) | 0 | 812.4 |
| L-Phenylalanine (mg) | 0 | 1839.6 |
| L-Proline (mg) | 816 | 3061.2 |
| L-Serine (mg) | 928 | 1928.4 |
| L-Threonine (mg) | 0 | 2080.8 |
| L-Tryptophan (mg) | 183.2 | 1936.8 |
| L-Tyrosine (mg) | 629.12 | 1989.6 |
| L-Valine (mg) | 0 | 2160 |
| Taurine | 34.4 | 897.6 |

TABLE 9
Examples of Amino Acid Blends in Nutritional Compositions

| | | | | Blend 4 | |
|---|---|---|---|---|---|
| AA Blend/Serving | Blend 1 | Blend 2 | Blend 3 | (MMA/ | Blend 5 |
| (8.5 fl oz.) | (PKU) | (MSUD) | (HCU) | PA) | (IVA) |
| Glycine (mg) | 689 | 1416 | 1500 | 107 | 2180 |
| L-Alanine (mg) | 423 | 2591 | 1070 | 2270 | 4340 |
| L-Arginine (mg) | 951 | 2085 | 1550 | 2178 | 1900 |
| L-Asparagine (mg) | 0 | 1477 | 0 | 0 | 0 |
| L-Aspartic Acid (mg) | 1761 | 0 | 1930 | 2900 | 1110 |
| L-Carnitine (mg) | 18 | 40 | 21 | 20 | 1030 |
| L-Cystine (mg) | 245.4 | 565 | 393.2 | 277.6 | 161.9 |
| L-Glutamic Acid (mg) | 0 | 0 | 0 | 0 | 0 |
| L-Histidine (mg) | 882 | 1475 | 930 | 1450 | 600 |
| L-Isoleucine (mg) | 1570 | 0 | 1430 | 68 | 630 |
| L-Leucine (mg) | 4700 | 0 | 2450 | 3130 | 0 |
| L-Lysine (mg) | 1830 | 1808 | 1620 | 2280 | 1440 |
| L-Methionine (mg) | 570 | 677 | 0 | 0 | 431 |
| L-Phenylalanine (mg) | 0 | 1533 | 1080 | 1420 | 1230 |
| L-Proline (mg) | 1700 | 2551 | 1760 | 1020 | 2150 |
| L-Serine (mg) | 1220 | 1607 | 1250 | 1480 | 1160 |
| L-Threonine (mg) | 1590 | 1734 | 1140 | 0 | 1050 |
| L-Tryptophan (mg) | 480 | 1614 | 480 | 613 | 229 |
| L-Tyrosine (mg) | 1549.7 | 1658 | 786.4 | 971.5 | 994.6 |
| L-Valine (mg) | 1800 | 0 | 1560 | 0 | 717 |
| Taurine | 75 | 748 | 50 | 57 | 43 |

TABLE 10
Examples of Amino Acid Blends

| AA | Blend 1 | | Blend 2 | | Blend 3 | | Blend 4 | | Blend 5 | |
|---|---|---|---|---|---|---|---|---|---|---|
| Blend/Serving | (PKU) | | (MSUD) | | (HCU) | | (MMA/PA) | | (IVA) | |
| (8.5 fl oz.) | MIN | MAX | MIN | MAX | MIN | MAX | MIN | MAX | MIN | MAX |
| Glycine (mg) | 551.2 | 826.8 | 1133 | 1699 | 1200 | 1800 | 85.6 | 128.4 | 1744 | 2616 |
| L-Alanine (mg) | 338.4 | 507.6 | 2073 | 3109 | 856 | 1284 | 1816 | 2724 | 3472 | 5208 |
| L-Arginine | 760.8 | 1141.2 | 1668 | 2502 | 1240 | 1860 | 1742.4 | 2613.6 | 1520 | 2280 |
| (mg) | | | | | | | | | | |
| L-Asparagine | 0 | 0 | 1182 | 1772 | 0 | 0 | 0 | 0 | 0 | 0 |
| (mg) | | | | | | | | | | |
| L-Aspartic Acid | 1408.8 | 2113.2 | 0 | 0 | 1544 | 2316 | 2320 | 3480 | 888 | 1332 |
| (mg) | | | | | | | | | | |
| L-Carnitine | 14.4 | 21.6 | 32 | 48 | 16.8 | 25.2 | 16 | 24 | 824 | 1236 |
| (mg) | | | | | | | | | | |
| L-Cystine (mg) | 196.3 | 294.5 | 452 | 678 | 314.6 | 471.8 | 222.1 | 333.1 | 129.5 | 194.3 |
| L-Glutamic | 0 | 0 | 0 | 0 | 0 | 0 | 0 | 0 | 0 | 0 |
| Acid (mg) | | | | | | | | | | |
| L-Histidine | 705.6 | 1058.4 | 1180 | 1770 | 744 | 1116 | 1160 | 1740 | 480 | 720 |
| (mg) | | | | | | | | | | |
| L-Isoleucine | 1256 | 1884 | 0 | 0 | 1144 | 1716 | 54.4 | 81.6 | 504 | 756 |
| (mg) | | | | | | | | | | |
| L-Leucine (mg) | 3760 | 5640 | 0 | 0 | 1960 | 2940 | 2504 | 3756 | 0 | 0 |
| L-Lysine (mg) | 1464 | 2196 | 1446 | 2170 | 1296 | 1944 | 1824 | 2736 | 1152 | 1728 |
| L-Methionine | 456 | 684 | 541.6 | 812.4 | 0 | 0 | 0 | 0 | 344.8 | 517.2 |
| (mg) | | | | | | | | | | |
| L-Phenylalanine | 0 | 0 | 1226 | 1840 | 864 | 1296 | 1136 | 1704 | 984 | 1476 |
| (mg) | | | | | | | | | | |
| L-Proline (mg) | 1360 | 2040 | 2041 | 3061 | 1408 | 2112 | 816 | 1224 | 1720 | 2580 |
| L-Serine (mg) | 976 | 1464 | 1286 | 1928 | 1000 | 1500 | 1184 | 1776 | 928 | 1392 |
| L-Threonine | 1272 | 1908 | 1387 | 2081 | 912 | 1368 | 0 | 0 | 840 | 1260 |
| (mg) | | | | | | | | | | |
| L-Tryptophan | 384 | 576 | 1291 | 1937 | 384 | 576 | 490.4 | 735.6 | 183.2 | 274.8 |
| (mg) | | | | | | | | | | |
| L-Tyrosine | 1239.8 | 1859.6 | 1326 | 1990 | 629.12 | 943.68 | 777.2 | 1165.8 | 795.7 | 1193.5 |
| (mg) | | | | | | | | | | |
| L-Valine (mg) | 1440 | 2160 | 0 | 0 | 1248 | 1872 | 0 | 0 | 573.6 | 860.4 |
| Taurine | 60 | 90 | 598.4 | 897.6 | 40 | 60 | 45.6 | 68.4 | 34.4 | 51.6 |

TABLE 11
Nutrients

| | HCU | | IVA | | MMA-PA | | MSUD | | PKU | |
|---|---|---|---|---|---|---|---|---|---|---|
| | HOMACTIN | | ISOVACTIN | | PROMACTIN | | VILACTIN | | PHENACTIN | |
| | AA | | AA | | AA | | AA | | AA | |
| | 8.5 FL OZ | PER | 8.5 FL OZ | PER | 8.5 FL OZ | PER | 8.5 OZ | PER | 8.5 FL OZ | PER |
| Nutrients | (250 ML) | 100 ML | (250 ML) | 100 ML | (250 ML) | 100 ML | (250 ML) | 100 ML | (250 ML) | 100 ML |
| Calories From Fat | 57 | 23 | | | 58 | 23 | 58 | 23 | 57 | 23 |
| Protein Equivalent, g | 20 | 8 | | | 20 | 8 | 20 | 8 | 20 | 8 |
| Free Amino Acids, g | 23 | 9 | 24 | 9 | 23 | 9 | 24 | 10 | 24 | 9 |
| Carbohydrates, g | 11 | 4 | 11 | 4 | 11 | 4 | 11 | 4 | 11 | 4 |
| Sugar, g | 3 | 1 | 3.2 | 1 | 3.2 | 1 | 3.2 | 1 | 3 | 1 |
| Sugar Alcohols, g | 0 | 0 | 0 | 0 | | | 0 | 0 | 0 | 0 |
| Dietary Fiber, g | 2.8 | 1 | 2.8 | 1 | 2.8 | 1 | 2.8 | 1 | 2.8 | 1 |
| Fat, g | 6.4 | 2.6 | 6.3 | 2.5 | 6.5 | 2.6 | 6.5 | 2.6 | 6 | 2.4 |
| Saturated Fat, g | 0.5 | 0.2 | 0.5 | 0.2 | 0.7 | 0.3 | 0.7 | 0.3 | 0.5 | 0.2 |
| Trans Fat, g | 0.0 | 0.0 | 0.0 | 0.0 | 0.0 | 0.0 | 0.0 | 0.0 | 0.0 | 0.0 |
| Cholesterol, mg | 0.4 | 0 | 0.4 | 0 | 0.4 | 0 | 0.3 | 0 | 0.4 | 0 |
| Vitamin A, IU | 1080.0 | 432.0 | 1080.0 | 432.0 | 1080.0 | 432.0 | 1080.0 | 432.0 | 1080.0 | 432.0 |
| Vitamin C, mg | 40.0 | 16.0 | 40.0 | 16.0 | 40.0 | 16.0 | 40.0 | 16.0 | 40.0 | 16.0 |
| Vitamin D, IU | 620.0 | 248.0 | 620.0 | 248.0 | 620.0 | 248.0 | 620.0 | 248.0 | 620.0 | 248.0 |
| Vitamin E, IU | 10.0 | 4.0 | 10.0 | 4.0 | 10.0 | 4.0 | 10.0 | 4.0 | 10.0 | 4.0 |
| Vitamin K1, mcg | 20.0 | 8.0 | 20.0 | 8.0 | 20.0 | 8.0 | 20.0 | 8.0 | 20.0 | 8.0 |
| Vitamin K2 (MK-7), mcg | 20.0 | 8.0 | 20.0 | 8.0 | 20.0 | 8.0 | 20.0 | 8.0 | 20.0 | 8.0 |
| Thiamin (B1), mg | 0.5 | 0.2 | 0.5 | 0.2 | 0.5 | 0.2 | 0.5 | 0.2 | 0.5 | 0.2 |
| Riboflavin (B2), mg | 0.5 | 0.2 | 0.5 | 0.2 | 0.5 | 0.2 | 0.5 | 0.2 | 0.5 | 0.2 |
| Niacin (B3), mg | 6.6 | 2.6 | 6.6 | 2.6 | 6.6 | 2.6 | 6.6 | 2.6 | 6.6 | 2.6 |
| Vitamin B6, mg | 0.5 | 0.2 | 0.5 | 0.2 | 0.5 | 0.2 | 0.5 | 0.2 | 0.5 | 0.2 |
| Folic acid, mcg | 186.0 | 74.4 | 186.0 | 74.4 | 186.0 | 74.4 | | | 186.0 | 74.4 |
| Vitamin B12, mcg | 1.0 | 0.4 | 1.0 | 0.4 | 1.0 | 0.4 | 1.0 | 0.4 | 1.0 | 0.4 |
| Pantothenic Acid (B5), mg | 2.4 | 1.0 | 2.4 | 1.0 | 2.4 | 1.0 | 2.4 | 1.0 | 2.4 | 1.0 |
| Biotin, mcg | 10.6 | 4.2 | 10.6 | 4.2 | 10.6 | 4.2 | 10.6 | 4.2 | 10.6 | 4.2 |
| Choline, mg | 209.0 | 83.6 | 209.0 | 83.6 | 209.0 | 83.6 | 209.0 | 83.6 | 209.0 | 83.6 |
| Calcium, mg | 700.0 | 280.0 | 700.0 | 280.0 | 700.0 | 280.0 | 700.0 | 280.0 | 700.0 | 280.0 |
| Chromium, mcg | 17.3 | 6.9 | 17.3 | 6.9 | 17.3 | 6.9 | 17.4 | 6.9 | 17.3 | 6.9 |
| Copper, mg | 0.2 | 0.1 | 0.2 | 0.1 | 0.2 | 0.1 | 0.2 | 0.1 | 0.2 | 0.1 |
| Iodine, mcg | 76.0 | 30.4 | 76.0 | 30.4 | 76.0 | 30.4 | 76.0 | 30.4 | 76.0 | 30.4 |
| Iron, mg | 8.0 | 3.2 | 8.0 | 3.2 | 8.0 | 3.2 | 8.0 | 3.2 | 8.0 | 3.2 |
| Magnesium, mg | 160.0 | 64.0 | 160.0 | 64.0 | 160.0 | 64.0 | 160.0 | 64.0 | 160.0 | 64.0 |
| Manganese, mg | 1.0 | 0.4 | 1.0 | 0.4 | 1.0 | 0.4 | 1.0 | 0.4 | 1.0 | 0.4 |
| Molybdenum, mcg | 20.0 | 8.0 | 20.0 | 8.0 | 20.0 | 8.0 | 20.0 | 8.0 | 20.0 | 8.0 |
| Phosphorus, mg | 695.0 | 278.0 | 695.0 | 278.0 | 695.0 | 278.0 | 695.0 | 278.0 | 695.0 | 278.0 |
| Selenium, mcg | 27.0 | 10.8 | 27.0 | 10.8 | 27.0 | 10.8 | 26.6 | 10.6 | 27.0 | 10.8 |
| Zinc, mg | 5.0 | 2.0 | 5.0 | 2.0 | 5.0 | 2.0 | 5.0 | 2.0 | 5.0 | 2.0 |
| Potassium, mg | 500.0 | 200.0 | 500.0 | 200.0 | 500.0 | 200.0 | 500.0 | 200.0 | 500.0 | 200.0 |
| Sodium, mg | 233.0 | 93.2 | 233.0 | 93.2 | 233.0 | 93.2 | 233.0 | 93.2 | 233.0 | 93.2 |
| Chloride, mg | 20.0 | 8.0 | 20.0 | 8.0 | 20.0 | 8.0 | 432.0 | 172.8 | 20.0 | 8.0 |
| Inositol, mg | 0.0 | 0.0 | 0.0 | 0.0 | 0.0 | 0.0 | 0.0 | 0.0 | 0.0 | 0.0 |
| L-alanine, mg | 1070.0 | 428.0 | 4340.0 | 1736.0 | 2270.0 | 908.0 | 2160.0 | 864.0 | 423.0 | 169.2 |
| L-arginine, mg | 1550.0 | 620.0 | 1900.0 | 760.0 | 2178.0 | 871.2 | 1020.0 | 408.0 | 951.0 | 380.4 |
| L-asparagine, mg | | | | | 0.0 | 0.0 | 1160.0 | 464.0 | | |
| L-aspartate, mg | 1930.0 | 772.0 | 1110.0 | 440.0 | 2900.0 | 1160.0 | 0.0 | 0.0 | 1760.0 | 704.0 |
| L-cystine, mg | 810.0 | 324.0 | 220.0 | 88.0 | 860.0 | 344.0 | 440.0 | 176.0 | 777.0 | 310.8 |
| L-glutamate, mg | | | | | | | 700.0 | 280.0 | | |
| L-glycine, mg | 1500.0 | 600.0 | 2180.0 | 872.0 | 107.0 | 42.8 | 540.0 | 216.0 | 689.0 | 275.6 |
| L-histidine, mg | 930.0 | 372.0 | 600.0 | 240.0 | 1450.0 | 580.0 | 1420.0 | 568.0 | 882.0 | 352.8 |
| L-isoleucine, mg | 1430.0 | 572.0 | 630.0 | 252.0 | 68.0 | 27.2 | 0.0 | 0.0 | 1570.0 | 628.0 |
| L-leucine, mg | 2450.0 | 980.0 | 0.0 | 0.0 | 3130.0 | 1252.0 | 0.0 | 0.0 | 4700.0 | 1880.0 |
| L-lysine, mg | 1620.0 | 648.0 | 1440.0 | 576.0 | 2280.0 | 912.0 | 1820.0 | 728.0 | 1830.0 | 732.0 |
| L-methionine, mg | 0.0 | 0.0 | 431.0 | 172.4 | 0.0 | 0.0 | 600.0 | 240.0 | 570.0 | 228.0 |
| L-phenylalanine, mg | 1080.0 | 432.0 | 1230.0 | 492.0 | 1420.0 | 568.0 | 1760.0 | 704.0 | 0.0 | 0.0 |
| L-proline, mg | 1760.0 | 704.0 | 2150.0 | 860.0 | 1020.0 | 408.0 | 1180.0 | 472.0 | 1700.0 | 680.0 |
| L-serine, mg | 1250.0 | 500.0 | 1160.0 | 464.0 | 1480.0 | 592.0 | 900.0 | 360.0 | 1220.0 | 488.0 |
| L-threonine, mg | 1140.0 | 456.0 | 1050.0 | 420.0 | 0.0 | 0.0 | 1440.0 | 576.0 | 1590.0 | 636.0 |
| L-tryptophan, mg | 480.0 | 192.0 | 229.0 | 91.6 | 613.0 | 245.2 | 1060.0 | 424.0 | 480.0 | 192.0 |
| L-tyrosine, mg | 1410.0 | 564.0 | 1240.0 | 496.0 | 1420.0 | 568.0 | 1760.0 | 704.0 | 2250.0 | 900.0 |
| L-valine, mg | 1560.0 | 624.0 | 717.0 | 286.8 | 0.0 | 0.0 | 0.0 | 0.0 | 1800.0 | 720.0 |

1. A composition comprising: Vitamin D, calcium, phosphorus, zinc, magnesium, and Vitamin K, wherein the Vitamin D is between about 500 IU and about 770 IU of the total weight of the composition, calcium is between about 36% and about 56% of the total weight of the composition, phosphorus is between about 31% and about 48% of the total weight of the composition, zinc is between about 0.25% and about 0.40% of the total weight of the composition, magnesium is between about 8% and about 13% of the total weight of the composition, and Vitamin K is between about 1.2% and about 3.5% of the total weight of the composition.

2. A composition comprising Vitamin D, calcium, phosphorus, zinc, magnesium, and Vitamin K, wherein in about 1.5 g to about 4.0 g of composition, Vitamin D is between about 500 IU and 770 IU, calcium is between about 500 mg and about 840 mg, phosphorus is between about 475 mg and about 720 mg, zinc is between about 4 mg and about 6 mg, magnesium is between about 125 mg and about 195 mg, and Vitamin K is between about 20 μg and about 50 μg.

3-4. (canceled)

5. The composition of claim 1, further comprising inulin.

6. (canceled)

7. The composition of claim 5, wherein from about 0 g to about 2.5 g inulin is present in 3.5 g of the composition.

8-9. (canceled)

10. A composition comprising a composition of claim 1 and a protein source, wherein Vitamin D is between about 500 IU per 20 g Protein Equivalent (PE) and 770 IU per 20 g PE, calcium is between about 500 mg per 20 g PE and about 840 mg per 20 g PE, phosphorus is between about 475 mg per 20 g PE and about 720 mg per 20 g PE, zinc is between about 4 mg per 20 g PE and about 6 mg per 20 g PE, magnesium is between about 125 mg per 20 g PE and about 195 mg per 20 g PE, and the Vitamin K is between about 20 μg per 20 g PE and about 50 μg per 20 g PE.

11. The composition of claim 10, wherein the protein source comprises at least six amino acids selected from the group consisting of: histidine, isoleucine, leucine, lysine, methionine, phenylalanine, threonine, tryptophan, and valine.

12. A nutritional composition for dietary management of an inborn error of metabolism comprising: a protein source, a fat source, a carbohydrate source, and the composition of claim 1, and, wherein the protein source comprises at least six free amino acids.

13. The nutritional composition of claim 12, wherein the pH of the composition is from about pH 5.0 to about pH 6.0.

14-15. (canceled)

16. The nutritional composition of claim 12, wherein glycine ranges from about 85 mg/20 g PE to about 2510 mg/20 g PE, alanine ranges from about 360 mg/20 g PE to about 5000 mg/20 g PE, arginine ranges from about 435 mg/20 g PE to about 2500 mg/20 g PE, asparagine ranges from about 0 mg/20 g PE to about 2070 mg/20 g PE, aspartic acid ranges from about 0 mg/20 g PE to about 2900 mg/20 g PE, cystine ranges from about 190 mg/20 g PE to about 865 mg/20 g PE, glutamic acid ranges from about 0 mg/20 g PE to about 400 mg/20 g PE, glutamine ranges from about 195 mg/20 g PE to about 2870 mg/20 g PE, histidine ranges from about 510 mg/20 g PE to about 1450 mg/20 g PE, isoleucine ranges from about 0 mg/20 g PE to about 1570 mg/20 g PE, leucine ranges from about 0 mg/20 g PE to about 4710 mg/20 g PE, lysine ranges from about 775 mg/20 g PE to about 2300 mg/20 g PE, methionine ranges from about 0 mg/20 g PE to about 600 mg/20 g PE, phenylalanine ranges from about 0 mg/20 g PE to about 1500 mg/20 g PE, proline ranges from about 500 mg/20 g PE to about 2160 mg/20 g PE, serine ranges from about 380 mg/20 g PE to about 1485 mg/20 g PE, threonine ranges from about 0 mg/20 g PE to about 1600 mg/20 g PE, tryptophan ranges from about 195 mg/20 g PE to about 615 mg/20 g PE, tyrosine ranges from about 375 mg/20 g PE to about 2260 mg/20 g PE, and valine ranges from about 0 mg/20 g PE to about 1800 mg/20 g PE.

17. The nutritional composition of claim 12, further comprising at least one component selected from the group consisting of carnitine, taurine, and Docosahexaenoic acid (DHA).

18. The nutritional composition of claim 12, further comprising at least one vitamin selected from the group consisting of: Vitamin A, Vitamin E, biotin, folic acid, niacin, pantothenic acid, Vitamin B1, Vitamin B12, Vitamin B2, Vitamin B6, and Vitamin C.

19. (canceled)

20. The nutritional composition of claim 12, further comprising at least one mineral is selected from the group consisting of: chromium, copper, iodine, iron, manganese, molybdenum, potassium, selenium, and sodium.

21. The nutritional composition of claim 20, wherein calcium ranges from about 550 mg/20 g PE to about 840 mg/20 g PE, chromium ranges from about 13.5 μg/20 g PE to about 20.9 μg/20 g PE, copper ranges from about 0.15 mg/20 g PE to about 0.25 mg/20 g PE, iodine ranges from about 60 μg/20 g PE to about 91.5 μg/20 g PE, iron ranges from about 6 mg/20 g PE to about 9.75 mg/20 g PE, magnesium ranges from about 125 mg/20 g PE to about 195 mg/20 g PE, manganese ranges from about 0.75 mg/20 g PE to about 1.25 mg/20 g PE, molybdenum ranges from about 15 μg/20 g PE to about 25 μg/20 g PE, phosphorus ranges from about 475 mg/20 g PE to about 720 mg/20 g PE, potassium ranges from about 375 mg/20 g PE to about 520 mg/20 g PE, selenium ranges from about 21 μg/20 g PE to about 32 μg/20 g PE, sodium ranges from about 100 mg/20 g PE to about 200 mg/20 g PE, and zinc ranges from about 4 mg/20 g PE to about 6 mg/20 g PE.

22-30. (canceled)
